(19) United States
(12) Patent Application Publication
(10) Pub. No.: US 2005/0244907 A1
(43) Pub. Date: Nov. 3, 2005

(54) Methods, compositions, and kits for analysis of enzyme activity in cells

(75) Inventors: Ronald J. Graham, San Ramon, CA (US); Michael Sekar, Palo Alto, CA (US); Maura Barbisin, San Mateo, CA (US)

Correspondence Address: DECHERT LLP, P.O. BOX 10004, PALO ALTO, CA, 94303, US

(73) Assignee: Applera Corporation

(21) Appl. No.: 11/014,447

(22) Filed: Dec. 15, 2004

Related U.S. Application Data
(60) Provisional application No. 60/529,953, filed on Dec. 15, 2003.
(60) Provisional application No. 60/542,425, filed on Feb. 6, 2004.

(51) Int. Cl. C12Q 1/68; C12Q 1/66

(52) U.S. Cl. 435/8

(57) ABSTRACT

In one aspect, the present disclosure relates to methods for detecting an activity of an enzyme in a cell. In some embodiments, the methods include contacting a cell with a liposome containing at least one substrate thereby facilitating introduction of the substrate into the cell. The substrate is capable of producing a detectable light signal when acted on by the enzyme, and the signal is detected. The methods can be used in screening agents that can inhibit or activate an enzyme activity. The methods can also be used in various downstream assays such the detection of interactions between intracellular proteins, screening for variants of an enzyme, and detection of various diseases. Compositions and kits for carrying out the various methods are also provided.

### 1. CROSS-REFERENCE TO RELATED APPLICATIONS

This application claims benefit under 35 U.S.C. §119(e) to application Ser. No. 60/529,953, entitled “Methods and Compositions for Analysis of Enzyme Activity in Cells,” filed Dec. 15, 2003 and application Ser. No. 60/542,425, entitled “Methods, Compositions, and Kits for Analysis of Enzyme Activity in Cells,” filed Jan. 6, 2004.

### 2. FIELD

The disclosure generally concerns methods and compositions useful in the identification and/or analysis of enzyme activity in cells.

### 3. INTRODUCTION

The study of enzyme activity within cells has been conducted using a variety of methods. In one method, the cell membrane is broken to create a cytosolic extract of cellular components including the enzyme which is the object of study. Various tests are performed on the cytosolic extract or on the purified enzyme to determine the enzyme activity. However, the preparation of extracts or purified enzymes typically involves destruction of the cell. Because enzymes are frequently bound in highly organized enzyme pathways, the disruption and death of the cell can greatly affect enzyme activity. In other methods, the enzyme activity is determined from the study of extracellular events, such as the presence or lack of the products of enzyme activity. Such methods are indirect, and may not accurately reflect the activity of the enzyme.

Other strategies involve the introduction of enzyme substrates into the cell itself. There is particular interest in using enzyme substrates that are linked to fluorescent compounds such that the substrates undergo cleavage by the enzyme with release of the fluorescent compound. However, such fluorescent substrates are usually of considerable size and very few are able to penetrate the outer membrane of the cell. A number of strategies have been employed to introduce these fluorescent substrates into the cytosol. Examples comprise hypotonic shock in which the cell is exposed at an elevated temperature (e.g., 37° C.) to a hypotonic solution to induce leakiness of the membrane and allow entry of the substrate; electroporation; and microinjection. These techniques can damage the cell membrane and alter its permeability, thus allowing the substrate, and other molecules to leak from the cell. Moreover, these methods are physically disruptive to the cell and can lead to drastic alteration its overall biochemistry or, in many cases, cell death.

### 4. SUMMARY

In one aspect, methods are provided for detecting an activity of an enzyme in a cell. In some embodiments, the method involves contacting the cell with a liposome that contains (i.e. encapsulates) at least one substrate that is capable of producing a detectable light signal when acted on by the enzyme, and detecting the amount of a light signal in the cell wherein the amount indicates a level of the enzyme activity in the cell. Encapsulation in a liposome facilitates intracellular delivery of substrate. Delivery is faster and/or in greater yield (on a concentration basis) than when the unencapsulated substrate is used. The detectable light signal can be a fluorescent signal or a chemiluminescent signal, for example.

Another aspect concerns the analysis of agents that can modulate the activity of an enzyme in a cell. In some embodiments, a cell can be exposed to an agent such as a known or suspected inhibitor or activator of the enzyme. The cell is then contacted with one or more liposomes encapsulating a substrate that is capable of producing a detectable light signal upon chemical modification by an intracellular enzyme in order to determine the effect of the agent on the enzyme activity. Kinetic parameters of an intracellular enzyme can be studied by determining the rate of change of a detectable light signal at various levels of substrate, for example.

Still other aspects involve assays for the analysis of intra-cellular enzyme activity. In some embodiments, using various genetic techniques, a library of cells can be prepared having different variants of an enzyme of interest, and these can be assayed using liposome-encapsulated substrates as indicated herein. In some embodiments, a substrate is introduced into a cell that comprises a reporter system for detecting protein-protein interactions. In some embodiments, the system comprises fusion proteins between putative binding proteins and two or more inactive, weakly-complementing mutants of a reporter enzyme (e.g., β-galactosidase or β-lactamase). Association between the proteins of interest brings the complementing reporter mutants into proximity so that complementation occurs and active reporter enzyme is produced. The active enzyme is capable of acting on the substrate to produce a fluorescent or chemiluminescent product. The method can be used to screen for agents that modulate the interaction between the binding proteins of interest.

In other aspects, the disclosure concerns compositions and kits for carrying out the methods described herein.

### 5. BRIEF DESCRIPTION OF THE DRAWINGS

The skilled artisan will understand that the drawings, described below, are for illustration purposes only. The drawings are not intended to limit the scope of the present teachings in anyway.

FIG. 1A and FIG. 1B illustrate a fluorescence image of psi 2 BAGα cells after exposure to liposomes containing DDAO-gal.

FIG. 2A and FIG. 2B illustrate a fluorescence image of psi 2 BAGα cells after exposure to DDAO-gal not contained within liposomes.

FIG. 3A and FIG. 3B illustrate a fluorescence image of psi 2 BAGα cells after exposure to liposomes containing DDAO.

FIG. 4A and FIG. 4B illustrate a fluorescence image of HeLa cells after exposure to liposomes containing DDAO-gal.

FIG. 5A and FIG. 5B illustrate a fluorescence image of HeLa cells after exposure to DDAO-gal not contained within liposomes.

FIG. 6A and FIG. 6B illustrate a fluorescence image of HeLa cells after exposure to liposomes containing DDAO.

FIG. 7A and FIG. 7B illustrate a fluorescence image of CHO cells after exposure to liposomes containing DDAO-gal.

FIG. 8A and FIG. 8B illustrate a fluorescence image of CHO cells after exposure to DDAO-gal not contained within liposomes.

FIG. 9A and FIG. 9B illustrate a fluorescence image of CHO cells after exposure to liposomes containing DDAO.

### 6. DESCRIPTION OF VARIOUS EMBODIMENTS

In one aspect, the present disclosure relates to methods for detecting an activity of one or more enzymes in a cell. In some embodiments, a cell is contacted with a liposome containing a substrate capable of producing a detectable light signal when acted upon by the enzyme, and detecting the amount of a light signal in the cell, wherein the amount indicates a level of the enzyme activity in the cell.

A wide variety of enzymes can be detected, and generally comprise any protein that is capable of chemically modifying or altering a substrate that is capable of producing a detectable light signal. A detectable light signal can be for example, a chromogenic, fluorescent, phosphorescent or chemiluminescent signal, such as a detectable product of an enzymatic reaction catalyzed by an enzyme. The wavelength of the light signal can be any detectable wavelength, ranging, for example, from ultraviolet, visible, through infrared. Photoluminescence is the process whereby a material is induced to luminesce when it absorbs electromagnetic radiation. Fluorescence and phosphorescence are types of photoluminescence. Chemiluminescence is the process whereby energy is released from a material in the form of light because of a chemical reaction (or reactions) and requires no light source for excitation (as in fluorescence and phosphorescence). Non-limiting examples of enzymes that can be detected comprise β-glucuronidase, carboxylesterase, lipases, phospholipases, sulphatases, ureases, peptidases, sulfatases, thioesterases, and proteases. In some embodiments, the enzyme is a hydrolytic enzyme. Non-limiting examples of hydrolytic enzymes comprise alkaline and acid phosphatases, esterases, decarboxylases, phospholipase D,P-xylosidase, β-fucosidase, thioglucosidase, α-galactosidase, α-glucosidase, β-glucosidase, β-glucuronidase, α-mannosidase, β-mannosidase, β-fructofuranosidase, β-glucosiduronase, and trypsin. Other examples of enzymes that can be studied comprise hydrolases, oxidoreductases, saccharidases, β-glucosidase, β-lactamases, β-glucuronidase, α-galactosidase, β-hexosaminidase, cholesterol esterase, nucleases, arylsulfatase, phospholipase, caspase 3, luciferases, and phosphatase. Specific examples of enzymes comprise E. coli β-glucosidase, E. coli TME-1 β-lactamase, glutathione-S-transferase, chloramphenicol acetyltransferase (CAT), uricase, secreted form of human placental alkaline phosphatase (SEAP), dihydrofolate reductase (DHFR), protein kinase A (PKA), protein kinase (PKC) isozymes (e.g., PKCα, PKCβ and PKCγ). Other examples, comprise fatty acid synthase, cysteine protease, and phospholipase A2.

Additional non-limiting examples of enzyme activities that can be detected using the present methods include the following: estrogen sulfotransferase (SULT 1E); estrone sulfatase (E.C. 3.1.6.2.) as assayed using a substrate such as substrate 3,4-benzocoumarin 7-O-sulfate (see Bilban, et al., 2000, Bioorganic and Medicinal Chemistry Letters 10:967-969; farnesyl:protein transferase which can be detected using a substrate such as N-dansyl-GCVLS (see Pompliano, et al., 1992, J. Am. Chem. Soc. 114:7945-7946); sialyl transferase (E.C. 2.4.99.1) which can utilize a glycosyl donor such as Nap-CMP-NANA or a glycosyl acceptor such as LacNAc-Dan (see Washiya, et al., 2000, Analytical Biochemistry 283:39-48); histone deacetylase which can be detected using a substrate such as MAL (Sigma catalog no. H 9660); caspase 8 which can be monitored using a substrate such as Z-IETD-R110 (Molecular Probes catalog no. A-22125); and selected cytochrome P450 isozymes which oxidize substrates such as ethoxyresorufin (Sigma catalog no. CYTO-1A). Further non-limiting examples of enzymes that can be analyzed using the instant methods include: protein kinases, estrogen sulfotransferases, carbohydrate sulfotransferases, tyrosylprotein sulfotransferases, farnesyl transferases, COX-1, 2, dihydrofolate reductase, aromatase, alcohol dehydrogenase, acetylcholinesterase, sialyl transferase, adenylyl cyclase, inositol phosphoceramide (IPC) synthase, glycosyl transferases, lanosterol 14α-demethylase, type 2 fatty acid synthase, thymidylate synthase, geranylgeranyl transferase, methionine synthase, serine hydroxymethyltransferase, HMG-CoA reductase, histone acetyltransferase, histone deacetylase, cyclic nucleotide phosphodiesterases, phosphoinositide 3 kinase, 17β-hydroxysteroid dehydrogenase, topoisomerase, telomerase, squalene synthase, palmatoyl transferase, myristoyl transferase, firefly luciferase (EC 1.13.12.7), and Rinella luciferase.

The analysis of a particular enzyme activity requires the use of a substrate that will be recognized and altered by one or more enzymes that exhibit that activity. A substrate can be designed and synthesized based upon the specificity of the enzyme. Alternatively, a substrate can be selected from a wide variety of substrates that are commercially available, or that can be prepared by known techniques. The substrate is preferably compatible with the cell such that the cell will remain metabolically active for at least the duration of the assay. In some embodiments, the substrate will have a leaving group and an indicator group. The leaving group can be selected for removal (e.g., via hydrolytic cleavage) by the enzyme to be analyzed. The indicator group can be selected for its ability to have a first state when joined to the leaving group, and a second state when the leaving group is removed from the indicator group by the enzyme. The first state must be detectably different from the second state, however, no particular degree of difference is required. In some embodiments, in the first state the indicator is less fluorescent than it is in the second state. In some embodiments, the indicator is fluorescent in both the first and second states, but has an emission profile in the first state that differs from the emission profile in the second state such that one or more emission wavelengths can be monitored in order to detect enzyme activity. In some embodiments, the indicator group is excitable at a wavelength within the visible range, for example, at wavelength between about 450 to 500 nanometers (nm). In some embodiments, the indicator group emits in the range of about 480 to 620 nm, 500 to 600 nm, or 500 to 550 nm. Auto-fluorescence of many cell types is most prevalent below about 500 nm, and an indicator that emits above this wavelength can be used in some embodiments in order to minimize this potential interference. In some embodiments, the indicator group can be selected or derived from fluorogenic and/or chemiluminescent compounds. Examples of suitable fluorogenic indicator compounds include xanthene compounds. Specific examples are rhodamine 110, rhodol, fluorescein, and various substituted derivatives thereof (e.g., see U.S. Pat. No. 5,871,946). In some embodiments, the indicator group is more fluorescent in the first state than in the second state. For example, a substrate can comprise a dye pair consisting of a donor and an acceptor (i.e., the indicator group) which are in close proximity in the first state. During the enzymatic reaction, the donor is cleaved away so that the fluorescence of the indicator drops in the second state. The dye pair can comprise a donor dye which absorbs light at a first wavelength and emits excitation energy in response, and acceptor dye which is capable of absorbing the excitation energy emitted by the donor dye and fluorescing at a second wavelength in response. A wide variety of dye pairs can be used (see, e.g., U.S. Pat. Nos. 5,800,996, 5,863,727, 5,945,526, 6,130,073 and 6,399,392). In some embodiments, the donor dye can be a member of the xanthene class of dyes, and the acceptor dye can be a member of the xanthene, cyanine, phthlaocyanine, or squaraine class of dyes. In some embodiments, the acceptor has an emission that is greater than about 600 nm or at least about 100 nm greater than the absorbance maximum of the donor dye. The members of the dye pair can be positioned in a substrate such that they can undergo energy transfer. Examples of such substrates labeled with dye pairs are described hereinbelow in relation to substrates of β-lactamase.

The leaving group can be selected according to the enzyme that is to be assayed. The leaving group will have utility for assaying many of the various cellular enzymes as described above. In non-limiting examples, the leaving group can be selected from amino acids, peptides, saccharides, sulfates, phosphates, esters, phosphate esters, nucleotides, polynucleotides, nucleic acids, pyrimidines, purines, nucleosides, lipids and mixtures thereof. In some embodiments, more than one leaving group can be attached to an indicator group. For example, a peptide leaving group and a lipid leaving group can be separately attached to a signal-producing compound such as rhodamine 110. Other leaving groups suitable for an enzyme to be assayed can be determined empirically or obtained from the literature. See, for example, Mentlein, et al., 1991, Eur. J. Clin. Chem. Clin. Biochem. 29:477-480; Schon, et al., 1987, Eur. J. Immunol. 17:1821-1826; Ferrer Lopez, et al., 1992, J. Lab. Clin. Med. 119:231-239; and Royer, et al., 1973, J. Biol. Chem. 248:1807-1812. In some embodiments, luminescent substrates comprising 1,2-dioxetane as indicator group (such as described in U.S. Pat. Nos. 6,660,529; 6,586,196; 6,514,717; 6,355,441; 6,287,767; and Re. 36,536) can be used as described herein. Many of these compounds are commercially available (Tropix, Inc., Bedford, Mass.) under the trademarks GALACTO-LIGHT™, GALACTO-LIGHT PLUS™, GALACTO STAR™, GUS-LIGHT™, PHOSPHA-LIGHT™, and DUAL-LIGHT®. Other suitable substrates include adamantine-dioxetanes such as 3-(2′-spiroadamantane)-4-methoxy-(3″-phosphoryloxy)phenyl-1,2-dioxetane disodium salt (AMPPD) and 3-(4-methoxyspiro[1,2 dioxetane-3,2′-tricyclo[3.3.1.1,3,7]decan]-4-yl)phenyl-β-d-galactopyranoside (AMPGD), which are substrates for alkaline phosphatase and β-galactosidase, respectively (see, e.g., Van Dyke et al. in: Luminescence Biotechnology Instruments and Applications, Van Dyke et al., eds. pages 3-29, CRC Press, 2002). These compounds are available commercially (Applied Biosystems, an Applera Corporation business) under the trademarks GALACTON®, GLUCON®, GLUCURON® and CSPD®.

In some embodiments, the substrate can be a β-galactosidyl substituted fluorogenic compound or substituted derivative thereof or a β-galactosidyl substituted fluorescein or substituted derivative thereof. Other examples of suitable substrates include 9H-(1,3-dichloro-9,9-dimethylacridin-2-one-7-yl) β-D-galactopyranoside, fluorescein di-β-D-galactoside, 2-nitrophenyl β-D-galactopyranoside, resorufin β-D-galactopyranoside, 6,8-difluoro-4-methylumbelliferyl β-D-galactopyranoside, β-methylumbelliferyl β-D-galactopyranoside, 3-carboxyumbelliferyl β-D-galactopyranoside, 5-chloromethylfluorescein di-β-D-galactopyranoside, and 5-(pentafluorobenzoylamino)fluorescein di-β-D-galactopyranoside.

In a particular example, the substrate is 9H-(1,3-dichloro-9,9-dimethylacridin-2-one-7-yl) β-D-galactopyranoside. In some embodiments, the substrate is fluorescein di-β-galactopyranoside (catalog no. F-1179, Molecular Probes, Eugene, Oreg.). In some embodiments, the substrate is 5-bromo-4-chloro-3-indoyl-β-galactopyranoside (X-gal).

In some embodiments, substrates for β-lactamase can be used. Examples of such substrates include those which include a fluorescent donor moiety and an acceptor (e.g., a fluorescence resonance energy transfer (FRET) dye pair) such as described in U.S. Pat. Nos. 5,955,604 and 6,031,094. Fluorescence energy transfer between the donor and quencher can be monitored as an indicator of β-lactamase activity. β-lactamase substrates have been described which include one or more attached groups (e.g., acetyl, butyryl, and acetoxymethyl) which makes them permeable through cell membranes wherein the attached group is hydrolytically cleaved by endogenous esterases after the substrate enters the cell (Klokamik, et al., 1998, Science, 279:84-88; Gao, et al., J. Am. Chem. Soc., 2003, 125:11146-11147; and PCT WO 96/30540 published Oct. 3, 1996). In some embodiments, the present methods utilize such substrates. However, in some embodiments, such substrates are used but which lack these attached groups.

In some embodiments, the following substrates of β-lactamase can be used: 5-Thia-1-azabicyclo[4.2.0]oct-2-ene-2-carboxylic acid, 8-oxo-3-[3-[(2-oxo-2H-1-benzopyran-7-yl)oxy]-1-propenyl]-7-[(phenylacetyl)amino]-, (6R,7R)-(9CI) (CC1, (CA Registry no. 609812-88-6)); 5-Thia-1-azabicyclo[4.2.0]oct-2-ene-2-carboxylic acid, 8-oxo-3-[3-[(2-oxo-2H-1-benzopyran-7-yl)oxy]-1-propenyl]-7-[(phenylacetyl)amino]-, 5-oxide, (6R,7R)-(9CI) (CC2, (CA Reg. No. 609812-89-7)); 5-Thia-1-azabicyclo[4.2.0]oct-2-ene-2-carboxylic acid, 8-oxo-3-[(1Z)-3-[(3-oxo-3H-phenoxazin-7-yl)oxy]-1-propenyl]-7-[(2-thienylacetyl)amino]-, 5-oxide, (6R,7R)-(9CI) (CR2, (CA Registry No. 452280-30-7)); and 5-Thia-1-azabicyclo[4.2.0]oct-2-ene-2-carboxylic acid, 7-[[[[(6-chloro-7-hydroxy-2-oxo-2H-1-benzopyran-3-yl)carbonyl]amino]acetyl]amino]-3-[[(3′,6′-dihydroxy-3-oxospiro[isobenzofuran-1(3H), 9′-[9H]xanthen]-5-yl)thio]methyl]-8-oxo-, (6R,7R)-(9CI) (CCF2, (CA Registry No. 183736-52-9)). In some embodiments, the following substrates can be used: 5-Thia-1-azabicyclo[4.2.0]oct-2-ene-2-carboxylic acid, 8-oxo-3-[3-[(3-oxo-3H-phenoxazin-7-yl)oxy]-1-propenyl]-7-[(2-thienylacetyl)amino]-, (acetyloxy)methyl ester, 5-oxide, (6R,7R)-(9CI) (CR2/AM, (CA Registry No. 452280-31-8)); 5-Thia-1-azabicyclo[4.2.0]oct-2-ene-2-carboxylic acid, 7-[[[[[7-[(acetyloxy)methoxy]-6-chloro-2-oxo-2H-1-benzopyran-3-yl]carbonyl]amino]acetyl]amino]-3-[[[3′,6′-bis(acetyloxy)-3-oxospiro[isobenzofuran-1 (3H), 9′-[9H]xanthen]-5-yl]thio]methyl]-8-oxo-, (acetyloxy)methyl ester, (6R,7R)-(9CI) (CCF2/AM, (CA Registry No. 183736-66-5)), or mixtures thereof.

In some embodiments, the following substrates of various luciferase enzymes can be used: varglin luciferin (catalog no. NF-CV-HBR, Nanolight Technology, Pinetop, Ariz.), coelenterazine (catalog no. NF-CTZ-FB, Nanolight; and see catalog no. E2810 and part no. TM055, Promega), firefly luciferin (D-(−)-2-(6′-hydroxy-2′-benzothiazolyl)thiazoline-4-carboxylic acid, available from Pierce Biotechnology, Molecular Probes, and Nanolight), cyprinda luciferin (catalog no. NF-CV-HBR, Nanolight), bacterial luciferin, dinoflagellate luciferin, or mixtures thereof.

In some embodiments, the following substrates of various cytochrome P450 isozymes can be used: luciferin 6′ chloroethyl ether (luciferin-CEE, catalog no. V8751, Promega, Madison, Wis.), luciferin 6′ methyl ether (luciferin-ME, catalog no. V8771, Promega), 6′ deoxyluciferin (luciferin H, catalog no. V8791, Promega), luciferin 6′ benzyl ether (luciferin-BE, catalog no. V8801, Promega), or mixtures thereof.

The present methods can be used in assays for enzymes that mediate the formation of an adduct with a substrate. In some embodiments, phophorylation activity can be monitored using the present methods. For example, a fluorescent-labeled oligopeptide (DACM-CLRRASLK-fluorescein), containing a consensus amino acid sequence (RRXSL) of cyclic AMP (cAMP) dependent protein kinase A (PKA) substrate-proteins (Ohuchi et al., 2000, Analyst 125:1905-1907; Ohuchi et al., 2001, Analytical Sci. (supp.) 17:i1465-i1467), can be used as a substrate in the present methods. The phosphorylation of the serine residue in the substrate causes a change in fluorescent intensity. Other suitable substrates include one or more members of the library of fluorescently-labeled PKC substrates described by Yeh et al. (J. Biol. Chem. 277:11527-11532) and fluorescent peptide substrates for PKC and PKA which contain a kinase sensing motif such as described by Shults et al. (2003, J. Am. Chem. Soc. 125:14248-14249). Non-limiting examples of suitable peptide substrates comprise one or more of the following (see Shults et al.):

(SEQ ID:1)

Ac-Sox-Pro-Gly-Ser*-Phe-Arg-Arg-Arg-NH₂;

(SEQ ID:2)

Ac-Leu-Arg-Arg-Ala-Ser*-Leu-Pro-Sox-NH₂;

(SEQ ID:3)

Ac-Sox-Pro-Gly-Thr*-Phe-Arg-Arg-Arg-NH₂;
and

(SEQ ID:4)

Ac-Sox-Pro-Gly-Ile-Tyr*-Ala-Ala-Pro-Phe-Ala-Lys-
Lys-Lys-NH₂.

The residue that is phosphorylated is marked (*). Sox is an amino acid having the formula 2-Quinolinepropanoic acid, α-amino-5-[(dimethylamino)sulfonyl]-8-hydroxy-, (αS)-(9CI) (CA Registry No. 639079-38-2; see Shults et al., 2003, J. Am. Chem. Assoc. 125:10591-10597).

In some embodiments, the substrate is membrane impermeable, or highly impermeable, and can be encapsulated in a liposome that is contacted with a cell.

In some embodiments, a liposome can contain at least two substrates, wherein the substrates are capable of producing distinguishable signals, such as two distinguishable fluorescent signals (see, e.g., U.S. Pat. No. 5,863,727). More than one substrate can be used, and can allow detection of one or more different enzyme activities.

The methods described above uses a substrate encapsulated in a liposome in order to introduce at least one substrate into the cell. Essentially any lipid complex that can encapsulate a substrate and facilitate delivery of the substrate as described herein can be used in the present methods, compositions and kits. An example of a suitable lipid complex includes liposomes. Essentially any liposome can be used in the methods so long as it is substantially non-toxic to the cell to which it is contacted, at least for the duration of the assay, and is capable of introducing the substrate into the cell under the conditions of the assay. Liposomes can be anionic, cationic or neutral depending upon the choice of hydrophilic group. For instance, when a lipid with a phosphate or a sulfate group is used in the liposome preparation, the resulting liposomes will be anionic. When amino-containing lipids are used, the liposomes will have a positive charge, and will be cationic. When polyethylenoxy or glycol groups are present in the lipid, neutral liposomes are obtained. Additional compounds suitable for forming liposomes can be found in “McCutchen's Detergents and Emulsifiers and McCutchen's functional materials”, Allured Publishing Company, Richwood, N.J., U.S.A.

Liposomes can be prepared using various methods (e.g., as described in Lasic, Liposomes in Gene Delivery, CRC Press, New York pp. 67-112 (1997), Ann. Rev. Biophys. Bioeng. 9:467-508 (1980); European Patent Application 0172007; U.S. Pat. Nos. 4,229,360; 4,241,046; 4,235,871; 5,455,157; 6,284,538; 6,458,381; and 6,534,018). Many preparation methods involve steps such as preparation of the lipid for hydration, followed by hydration with agitation (e.g., extrusion, sonication, and/or homogenization), and sizing to a homogeneous distribution of liposomes, for example, although any suitable preparation method can be used. Properties of liposomes can vary depending on the composition (cationic, anionic, neutral lipid species), however, the same preparation method can be used for all liposomes regardless of composition. Liposomes of various sizes and shapes can be used in the methods described herein. For example, large multilamellar vesicles (LMV) can be used, as can unilamellar vesicles such as small (SUV), large (LUV) or giant (GUV). A suitable liposomal preparation can comprise a liposomes a having heterogeneous size distribution. Alternatively, liposomes can be prepared having a narrow size distribution. For example, liposomes having a diameter in the range of about 50 nm to about 250 nm can be prepared, although other sizes are possible. In those embodiments in which substrates or other compounds are encapsulated into liposomes, conventional methods can be used for loading, such as reverse phase methods and sonication (e.g., as described by Lasic (1997) p. 93 and in U.S. Pat. No. 4,888,288). After loading, the liposomes can optionally be subjected to dialysis or molecular sieving (e.g. by Q Sepharose separation). Substrate can be encapsulated during liposome preparation, such as described in Examples herein. In some embodiments, the substrate is provided in a form that is at least 80%, 90%, 95% or 99% liposomal as opposed to free in solution. To enhance stability, a liposome preparation can optionally be stored in the dark, under argon, and at a low temperature, such as 4° C., for example.

A liposome preparation for use herein can include one or more of a variety of lipids, non-limiting examples of which include phosphatidic acid, phosphatidylethanolamine, phosphatidylcholine, phosphatidylinositols, phosphatidylglycerol, sphingomylelin, cardiolipin, lecithin, phosphatidylserine, cephalin, cerebrosides, dicetylphosphate, steroids, terpenes, acetylpalmitate, glycerol ricinoleate, hexadecyl stearate, isopropyl myristate, amphoteric polymers, triethanolamine lauryl sulfate and cationic lipids, 1-alkyl-2-acyl-phosphoglycerides, and 1-alkyl-1-enyl-2-acyl-phosphoglycerides. In some embodiments, the cationic lipids can include lipids having multiple hydroxy functionalities in the headgroup region, such as described by Banerjee et al. (J. Med. Chem., 2001, 44:4176-4185). In some embodiments, a cationic liposome preparation containing O,O′-ditetradecanoyl-N-(α-trimethylammonioacetyl)diethanolamine chloride, dioleoylphosphatidylethanolamine and cholesterol (e.g., in a molar ration of 4:3:3 as described by Serikawa, et al. Biochim. Biophys, Acta, 2000, 1467:419-430) can be used.

Other amphiphiles useful in forming liposomes include cationic lipids, such as described in Lasic (1997), pp. 81-86. In some embodiments, one or more of the following lipids can be used in preparing liposomes as described herein: dioctadecyl dimethyl ammonium bromide/chloride (DODAB/C), dioleoyloxy-3-(trimethylammonio)propane (DOTAP), stearylamine, dodecylamine, hexadecylamine, and dioctadecylammonium bromide. In some embodiments, the liposomes used herein will contain stearylamine at a mole % that is less than 20%, less than 10%, less than 5%, or less than 1%. In some embodiments, the liposomes are essentially devoid of stearylamine.

A wide variety of suitable lipids are commercially available (such as from Avanti Polar Lipids, Inc. Alabaster, Ala.). Liposome kits are commercially available (e.g., from Boehringer-Mannheim, ProMega, and Life Technologies (Gibco)). Non-limiting examples of suitable lipids include 1,2-dimyristoyl-sn-glycero-3-phosphate (Monosodium Salt) (DMPA•Na) (Avanti catalog no. 830845), 1,2-dimyristoyl-sn-glycero-3-phosphate (Monosodium Salt) (DOPS•Na) (Avanti catalog no. 830035), and 1,2-dioleoyl-3-trimethylammonium-propane (Chloride Salt) (DTOAP•Cl) (Avanti catalog no. 890890).

Other commercially available liposome kits include Lipofectin™, Lipofectamine™, Lipofectace™, CellFectin™, Transfectam™, TRX-50™, DC-Chol™ and DOSPER™ (e.g., as described in Lasic, p. 86).

The liposomes can also include synthetic lipid compounds such as D-erythro (C-18) derivatives including sphingosine, ceramide derivatives, and sphinganine; glycosylated (C18) sphingosine and phospholipid derivatives; D-erythro (C17) derivatives; D-erythro (C20) derivatives; and L-threo (C18) derivatives, all of which are commercially available (Avanti Polar Lipids).

Liposomes can include or be wholly formed from non-naturally occurring analogs of phospholipids that are resistant to lysis by certain phospholipases. In some embodiments of such analogs, the phosphate group is replaced by a phosphonate or phosphinate group (as described in U.S. Pat. No. 4,888,288). In addition, if the phospholipid normally includes an ester moiety (ester of a fatty acid), the ester linkage can be replaced with an ether linkage.

In some embodiments, lipophilic fluorescent dyes can be embedded non-covalently within the lipid phase of a liposome to assess the integrity of the liposome or to detect the fusion of the liposome with the cell outer membrane. Suitable examples of a lipophilic dye include 6-dodecanoyl-2-dimethylaminonaphthalene (LAURDAN) and 6-hexadecanoyl-2-(((2-(trimethylammonium)ethyl)methyl)-amino)naphthalene chloride (PATMAN) (see U.S. Pat. No. 6,569,631).

In some embodiments, a membrane impermeable fluorescent dye can be encapsulated along with substrate in a liposome and can act as a tracer to detect fusion and delivery of the liposomal contents into a cell. Examples of such tracer are rhodamine-dextran and fluorescently labeled inulin (e.g., see U.S. Pat. No. 6,423,547). Lipophilic dyes or tracers can be selected to have spectral characteristics that do not interfere with the detection of the substrates as described herein.

In some embodiments, fusigenic liposomes can be used. Fusigenic liposomes efficiently fuse with cellular membranes and can be prepared by coupling the fusion protein of hemagglutinin of Simian virus with liposomes subsequent to substrate encapsulation (e.g., see Dzau et al., 1996, Proc. Natl. Acad. Sci. USA 93:11421-11425).

Liposomes can include cholesterol. Cholesterol intercalates within the phosphatidylcholine bilayer with very little change in area by occupying the regions created by the bulky phosphatidylcholine headgroups. This increases the packing density and structural stability of the bilayer (New, R.R.C., 1990 In New, R.R.C. (ed): Liposomes: a practical approach, Oxford University Press, New York, pp 19-21). The concentration of cholesterol in liposomes can be in the range, for example, of about 5 to about 60 mol %, although higher or lower concentrations can be used.

The composition of the lipid mixture can be selected based on a variety of factors including cost, transition temperature of the lipids, stability during storage, and stability of the liposomes under the reaction conditions. The composition can be selected based upon the compatibility of the liposome with the cell being analyzed.

In some embodiments, lipids for forming liposomes are phospholipid-related materials, such as lecithin, lysolethicin, phosphatidylinositol, sphingomyelin, cephalin, cardiolipin, phosphatidic acid, cerebrosides, and dicetylphosphate. Additional non-phosphorous containing lipids include, e.g., acetyl palmitate, glycerol ricinoleate, hexadecyl stereate, isopropyl myristate, amphoteric acrylic polymers, alkylaryl sulfate polyethyloxylated fatty acid amides, and the like. In some embodiments, lipids can comprise one or more of: phosphatidylethanolamine, lysophosphatidylethanolamine, phosphatidylserine, stearylamine, dodecylamine, hexadecylamine, triethanolamine-lauryl sulfate.

Another type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

In some embodiments the liposome is cationic. In some embodiments, the liposome is cationic and includes a 1,2-diacyl-sn-glycero-3-alkylphosphocholine having the formula: 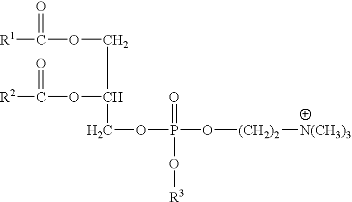
wherein:
R¹ is a saturated or unsaturated alkyl having from 6 to 30 carbon atoms;
R² is a saturated or unsaturated alkyl having from 6 to 30 carbon atoms;
R³ is a saturated or unsaturated alkyl having from 1 to 20 carbon atoms.

In some embodiments, a liposome can include 1,2-dioleoyl-sn-glycero-3-ethylphosphocholine. In some embodiments, the liposome can include both 1,2-dioleoyl-sn-glycero-3-ethylphosphocholine and 1,2-dioleoyl-sn-glycero-3-phosphocholine. In some embodiments, a liposome can include these two phospholipids in a molar ratio of about 1:1. In some embodiments, the liposome can include both 1,2-dioleoyl-sn-glycero-3-ethylphosphocholine and 1,2-dioleoyl-sn-glycero-3-phosphocholine in a molar ratio of about 1:2. The liposome can also include other lipids or components as described above.

In selecting lipids for preparing liposomes for use with various cells types as described hereinbelow, one or more tests can be performed to confirm that cells are viable after contact with the liposomes and under the condition of the enzyme assay. Any conventional test for viability can be used. For example, dye exclusion methods can be used. Trypan Blue is a blue stain which normally does not penetrate the plasma membrane and therefore is excluded from viable cells. Only cells with damaged plasma membrane are stained and take on a blue color. The stained and unstained cells can be counted in a hemacytometer with a standard light microscope and the percent of viability can be calculated. In another example, propidium iodide can be used in a similar manner. Propidium iodide is a DNA-RNA stain which, if the cell is dead and membranes are damaged, will penetrate the cell and stain the DNA-RNA. Non-viable cells become red and can be easily distinguished from the healthy, unstained cells with a fluorescent microscope or flow cytometer. Another dye that can be used is fluorescein diacetate-propidium iodide will cause living cells to take on a green color because the fluorescein diacetate which will be hydrolyzed, while dead cells become red from the propidium iodide (Breeuwer et al., 1995, Appl. Environ. Microbiol. 61:1614-1619; Widholm, 1972, Stain Technol. 47:189-194).

To confirm whether a particular liposomal preparation will be compatible for use in the detection methods herein, cells to be used in an assay method can be exposed to liposomes of a selected composition to determine if viability over the assay time period is affected. As used herein, “viable” refers to cells whose function and membrane structure are intact. For example, a Trypan Blue exclusion test can be performed at the end of an enzyme assay. In some embodiments, after the incubation of the cells (usually seeded at 100000 cells/ml the day before use) with a selected liposome preparation, a uniform suspension of cells can be prepared and diluted in Trypan Blue for a time period, such as a few seconds to a few minutes. The percent viability can be calculated. In some embodiments of such a test, if the viability of the cell sample decreases by 10% or more, then other lipid compositions can be prepared and tested. Cells can be tested for viability at any time point, such as before, during or after an enzyme assay, and any changes can be determined. In some embodiments, the viability of the cells after contact with a liposomal composition will decrease by less than 20%, less than 10%, less than 5% or less than 1%.

In some embodiments, cell viability can be checked by observation of cell morphology (e.g., with a standard light microscope). Cell morphology can be observed before and/or after contacting cells with a lipid complex, as described herein. For example, healthy HeLa cells appear polygonal and are adherent to the surface of the vessel in which they are contained, whereas damaged cells tend to shrink, roundup, detach and float in the medium. The number of cells that remain attached under a selected set of conditions can be used as another measure of toxicity. Cells can be further analyzed, such by use of a Trypan Blue exclusion test.

In some embodiments, a standard proliferation test can be used to investigate the cytotoxicity of a liposomal preparation. An exemplary test is the tetrazolium salt-based colorimetric test that detects viable cells exclusively. Living, metabolically active cells reduce tetrazolium salts to colored formazan compounds, whereas dead cells do not. This test can be performed in a microtitre plate after the treatment of cells with a selected liposome formulation. The colorimetric change in a sample versus control can be easily measured with a spectrophotometer. A cytotoxic factor will reduce the rate of tetrazolium salt cleavage by a population of cells.

The methods and compositions described herein can be used for investigations relating to a wide variety of cells. Cell types utilized in the methods, compositions, and kits disclosed include eukaryotic (e.g., animals, plants, yeast, fungi) and bacterial. Viable cells that can be used include fresh cells isolated from a living organism, cells grown or cultured in vitro, or cells reconstituted from frozen or freeze-dried preparations. Cells having a cell wall can be used after appropriate measures are taken to remove the cell wall (e.g., see Constabel, 1982, in “Plant Tissue Culture Methods” pp. 38-48, NRCC No. 19876, Nat. Res. Council of Canada, Saskatoon.). Further examples of cells which can be used are primary or established cell lines and other types of embryonic, neonatal or adult cells, or transformed cells (for example, spontaneously- or virally-transformed). These include, but are not limited to fibroblasts, macrophages, myoblasts, osteoclasts, osteoclasts, hematopoietic cells, neurons, glial cells, primary B- and T-cells, B- and T-cell lines, chondrocytes, keratinocytes, adipocytes and hepatocytes.

Cell lines which can be used in the methods of the present disclosure include, but are not limited to, those available from cell repositories such as the American Type Culture Collection (www.atcc.org), the World Data Center on Microorganisms (wdcm.nig.acjp), European Collection of Animal Cell Culture (www.ecacc.org) and the Japanese Cancer Research Resources Bank (cellbank.nihs.gojp). These cell lines include, but are not limited to, the following cell lines: 293, 293Tet-Off, CHO, CHO-AA8 Tet-Off, MCF7, MCF7 Tet-Off, LNCap, T-5, BSC-1, BHK-21, Phinx-A, 3T3, HeLa, psi Baga, PC3, DU145, ZR 75-1, HS 578-T, DBT, Bos, CV1, L-2, RK13, HTTA, HepG2, BHK-Jurkat, Daudi, RAMOS, KG-1, K562, U937, HSB-2, HL-60, MDAHB231, C2C12, HTB-26, HTB-129, HPIC5, A-431, CRL-1573, 3T3L1, Cama-1, J774A.1, HeLa 229, PT-67, Cos7, OST7, HeLa-S, THP-1, and NXA. Additional cell lines for use in the methods of the present disclosure can be obtained, for example, from cell line providers such as Clonetics Corporation (Walkersville, Md.; www.clonetics.com). Further non-limiting examples of suitable mammalian cells include Jurkat, HeLa, GHK-21, CHO-K1, COS7, COS, HepG2, PC12, 293, A431, A459, 1B, and L929 cells.

In some embodiments of carrying out enzyme detection and other methods as described herein, a cell suspension, or attached cells, are admixed with a suspension of liposomes encapsulating a substrate as described herein, the admixture is maintained for a time period and under physiological reaction conditions sufficient for the substrate to enter the cells and interact with the enzyme of interest and generate a detectable light signal.

Essentially any medium that is compatible with the cells under investigation and under the conditions of the method can be used. For example, a variety of cell culture media are described in “The Handbook of Microbiological Media” (Atlas and Parks, eds.) (1993, CRC Press, Boca Raton, Fla.). References describing the techniques involved in bacterial and animal cell culture include Sambrook et al., Molecular Cloning—A Laboratory Manual (2nd Ed.), Vol. 1-3 (1989, Cold Spring Harbor Laboratory, Cold Spring Harbor, N.Y.); Current Protocols in Molecular Biology, F. M. Ausubel et al., eds., Current Protocols, (a joint venture between Greene Publishing Associates, Inc. and John Wiley & Sons, Inc., supplemented through 2000); Freshney, Culture of Animal Cells, a Manual of Basic Technique, third edition (1994, Wiley-Liss, New York) and the references cited therein; Humason, Animal Tissue Techniques, fourth edition (1979, W.H. Freeman and Company, New York); and Ricciardelli, et al., 1989, In Vitro Cell Dev. Biol. 25:1016-1024. Information regarding plant cell culture can be found in Plant Cell and Tissue Culture in Liquid Systems, by Payne et al. (1992, John Wiley & Sons, Inc. New York, N.Y.); Plant Cell, Tissue and Organ Culture: Fundamental Methods by Gamborg and Phillips, eds. (1995, Springer Lab Manual, Springer-Verlag, Berlin), and is also available in commercial literature such as the Life Science Research Cell Culture Catalogue (1998) from Sigma-Aldrich, Inc (St Louis, Mo.) (Sigma-LSRCCC) and the Plant Culture Catalogue and supplement (1997) also from Sigma-Aldrich (Sigma-PCCS). Particular non-limiting examples of suitable media include conventional cell culture media. Such media are widely available (e.g., Sigma-Aldrich) and include Earle's Balanced Salts, Hanks' Balanced Salts, Tyrode's Salts and other salt mixtures.

The detection method can be performed using media containing serum. Non-limiting examples of suitable serum (all of which are commercially available, e.g., see Sigma-Aldrich) include fetal bovine serum (FBS), bovine serum, calf serum, newborn calf serum, goat serum, horse serum, human serum, chicken serum, porcine serum, sheep serum, serum replacements, embryonic fluid, and rabbit serum. In some embodiments, media comprising FBS in the range of about 2% to about 10%, in the range of about 4% to about 7%, and at a concentration of about 5%, can be used.

A suitable medium can include an aqueous medium having an osmolality, tonicity, pH value and ionic composition that supports and maintains cell viability. Exemplary media include normal saline, Ringer's solutions and commercially available cell culture media such as minimum essential medium (MEM), RPMI, Dulbecco's and Eagle's medium. One example of a suitable medium is buffered saline consisting of 4% v/v fetal calf serum, 10 mM Hepes, pH 7.2 at a temperature in the range of about 20° C.-37° C.

Methods described herein can be carried out under various reactions conditions. The reaction conditions can be selected to reduce or minimize adverse effects on the cell, and to not significantly interfere with the interaction of the liposomes with the cells or the detection of a light signal. In some embodiments, the conditions are essentially the same as those conventionally used to maintain viable cultures of cells. Reaction conditions can include selected values of temperature, pH value, osmolality, tonicity and the like. pH can range from a value of about 6.0 to a value of about 8.5 and, in certain cases from a value of about 6.5 to a value of about 7.5. The osmolality can range from about 200 milliosmols per liter (mOsm) to about 500 mOsm and, in some embodiments, from about 250 mOsm to about 350 mOsm. Tonicity can be maintained isotonic to the cells being used. The temperature during detection of enzyme activity can be maintained at any temperature that is compatible with the cells under investigation. In some embodiments the temperature can maintained at or near the membrane freezing point of the cell. In some embodiments, the temperature is above the membrane freezing point. In some embodiments, the temperature can be at least 4° C., 10° C., 15° C., at least 20° C., at least 30° C., at least 37° C., and at least 40° C. In some embodiments, the temperature can be selected in the range from about 10° C. to about 50° C. and, in some embodiments from about 20° C. to about 40° C.

Following contact with a liposome, a light detectable signal (e.g., a fluorescent or a chemiluminescent signal) in the cell is measured. A change (e.g., an increase or a decrease as compared with a control cell) in the light signal is indicative of enzyme activity. The light signal can be detected at one or more discrete time points following contact or, alternatively, the light signal can be detected substantially continuously as a function of time. Changes in light signal may be due to the activity of a single enzyme, or may be due to the cumulative activities of several different enzymes that have the same observable activity. In some cases, the different enzymes have different susceptibility toward inhibitors. For example, as described hereinbelow, a reporter gene encoding β-galactosidase can introduced into cells, followed by quantitation of the enzyme activity. However, an endogenous β-galactosidase is found in lysosomes in mammalian cells. The endogenous enzyme can be selectively inhibited, such as by chloroquine or 5% DMSO.

In the enzyme assay methods herein, the substrate is transferred into the cell in an amount suitable for generating a light detectable signal. No particular concentration of substrate is required as long as a signal can be detected. A suitable substrate concentration can be determined empirically, and cells that are known to possess the enzyme under study can be used as a basis for selecting such concentrations. Liposomes can be prepared using various concentrations of substrate and/or various amounts of liposomes can be used.

An enzyme assay as described herein can be used to measure the amount of an enzyme present in a cell. The rate of the reaction catalyzed by the enzyme can be determined by monitoring the progress of signal change with time. The signal is proportional to the amount of product formed. The rate of reaction is proportional to the amount of enzyme present, so that the rate of reaction provides a measure of the amount of enzyme present. The initial velocity of the enzyme reaction can be obtained as a function of the substrate concentration and various kinetic parameters obtained. The progress of a reaction can be monitored and analyzed (e.g., see U.S. Pat. No. 6,108,607 and Duggleby, 1995, Methods Enzymol. 249:60).

The present methods can be used for detecting the presence of an enzyme activity in a cell; screening for and/or quantifying an enzyme activity in a cell; determining apparent kinetic parameters, such as Km value or Vmax, of an enzyme with respect to selected substrates; detecting, screening for, and/or characterizing substrates of an enzyme; and detecting, screening for, and/or characterizing inhibitors, activators, and/or modulators of an enzyme. For example, in screening for an enzyme activity, a cell sample that contains, or can contain, a particular enzyme activity is contacted with liposome encapsulated substrate, and the light signal is measured to determine whether a change in light signal has occurred. Screening can be performed on numerous samples simultaneously in a multi-well or multi-reaction plate or device to increase the rate of throughput.

In another aspect, the disclosure concerns a method for analyzing modulators of an intracellular enzyme activity. In some embodiments, this involves contacting the cell with a modulator, such as an inhibitor or activator of the enzyme, contacting the cell with a liposome encapsulated substrate as described herein, and detecting a light signal. The method can be used for screening and/or pharmacological profiling of compounds modulating cellular enzymes. Non-limiting examples of such compounds can include extracellular signaling molecules, growth factors or differentiation factors, peptides, drugs or synthetic analogs, or the like, whose presence or effects might alter the activity of the enzyme of interest. Additional examples include antibiotics, anti-inflammatory drugs, neurotransmitters, growth hormones, or analgesics. The modulators investigated can be involved, for example, in regulating the activity of signal transduction pathways, cellular responses, cell surface receptors, ion channels, non-selective pores, second messenger pathways, signal transduction pathways, apoptosis, cellular necrosis or any other cellular responses. In some embodiments, mixtures of modulators can be utilized, for example, to detect synergistic effects. A modulator can act directly on the enzyme itself or can act indirectly, such as on another cellular target that can exert an effect on the enzyme. For example, if the enzyme catalyzes a reaction that is involved in a metabolic pathway or a signaling cascade, a modulator of an upstream reaction can influence the detected enzyme activity.

In studies of modulators, a substrate concentration that provides a linear rate will typically be used. For example, a substrate that is greater (e.g., 2- to 10-fold greater) than the Kₘ can be used. A substrate concentration that is transferred into the cell in an amount in excess of the amount of enzyme can also be used.

When a liposomal preparation is contacted with cells, the actual concentration of substrate in the cell may be unknown. For example, not all of the liposomes added to an incubation will fuse with the cells and there can be incomplete delivery of the encapsulated substrate. In some embodiments, a tracer, such as a fluorescent compound, can be included with the substrate in the liposome. The level of tracer can be used as a means to confirm delivery of the liposomal contents into the cell and to estimate the concentration of the substrate in the cell interior. In some embodiments, the level of tracer that is retained in a cell can be determined after a wash step to remove undelivered tracer. An example of a suitable tracer is fluorescently-labeled inulin, as mentioned above. In some embodiments, it can be assumed that the uptake of substrate is proportional to the uptake of tracer. Cellular volumes can be measured using conventional techniques, and the internal concentration of a tracer can be estimated and equated to the internal concentration of substrate. The concentration of substrate can then be correlated with the observed enzyme activity and various kinetic parameters, such as apparent Kₘ and Vₘₐₓ can be determined. To facilitate detection, the tracer can have a label that is distinguishable from that of the substrate or the substrate products resulting from reaction of substrate with enzyme.

When assessing various modulators, previously characterized inhibitors or activators can be used as reference compounds. For example, a number of inhibitors, both reversible and irreversible, exist for β-galactosidase. One example of an inhibitor that can be used as a reference is phenylethyl β-D-thiogalactopyranoside (PETG) (catalog no. P-1692, Molecular Probes) which is a competitive inhibitor of β-galactosidase.

The amount of the inhibitor will be determined by parameters such as the efficiency of transfer of the inhibitor across the membrane, the Kᵢ (or IC₅₀ value) of the inhibitor, and the enzyme activity in the cell. Therefore, the concentration of inhibitor in the external medium can vary widely and will usually be determined empirically. In many situations, a convenient concentration between 1 nM and 10 mM can be used. In some embodiments, the inhibitor or other modulator is contacted with the cell in the absence of liposomes prior to contact with the encapsulated substrate. In some embodiments, an inhibitor and a substrate are combined in the same liposome for transport across the membrane into the cell at the same time. Alternatively, an inhibitor and a substrate can be added via separate liposomes.

The detection methods described herein can be used in a wide variety of assays. For example, the methods and compositions can be used with cells comprising a reporter system, such as described by Blau (see, e.g., U.S. Pat. No. 6,342,345 and Proc. Natl. Acad. Sci. USA 93:12423-12427 (1996); PCT WO 96/30540 published Oct. 3, 1996) or such as described by Michnick (U.S. Pat. No. 6,270,964). When used in conjunction with a cells comprising a reporter system, the enzyme assay disclosed herein allows a broad range of studies of multiprotein and other types of multi-molecular interaction to be carried out quantitatively or qualitatively in living cells.

In some embodiments, the present enzyme detection method is used in conjunction with cells that include a reporter system. In some embodiments, the reporter system comprises a first β-galactosidase subunit coupled to a first putative binding protein; a second β-galactosidase subunit coupled to a second putative binding protein, wherein the first subunit and the second subunit are capable of associating to generate an enzymatically active complex capable of acting on the substrate to produce a fluorescent signal. The cell comprising the reporter system can be used to screen test agents that can modulate the interaction of the putative binding proteins. The interactions are mediated by the binding of the first protein and the second protein. This screening procedure comprises contacting a cell with a test agent and contacting the cell with a liposome containing a substrate thereby introducing said substrate into said cell, wherein said substrate is capable of producing a detectable light signal (e.g., a fluorescent or a chemiluminescent signal) when acted on by an enzyme. The screening procedure comprises detecting the level of a detectable light signal and comparing the level to a control cell not exposed to the test agent as an indication of the ability of the test agent to act as a modulator of interactions between the putative binding proteins. In some embodiments, the first β-galactosidase subunit coupled to the first putative binding protein comprises a fusion protein and the second β-galactosidase subunit coupled to a second putative binding protein comprises another fusion protein.

In some embodiments, the reporter system includes a first β-lactamase subunit coupled to a first putative binding protein; a second β-lactamase subunit coupled to a second putative binding protein, wherein the first subunit and the second subunit are capable of associating to generate an enzymatically active complex capable of acting on the substrate to produce a detectable light signal. An advantage of using β-lactamase activity as reporter is that mammalian cells do not express an endogenous β-lactamase activity. In contrast, β-glactosidaseis present in lysosomes and can interfere with certain enzyme assays.

In some embodiments, various other reporter systems can be used. For example, reporter subunits capable of complementation with low binding affinity can be derived from enzymes such as β-glucuronidase (GUS), alkaline phosphatase, peroxidase, chloramphenicol acetyltransferase (CAT) and luciferase.

A cell system such as described by Blau can also be used to screen for new binding partner(s) for a given target protein. For example, the target protein, fused to a weakly-complementing β-gal mutant can be stably expressed in a well-characterized cell line. Expression libraries containing cDNAs fused to a weakly-complementing β-gal mutant can be introduced into these cells using, for example, retroviral vectors (e.g., Kitamura et al., Proc Natl. Acad. Sci. USA 92:9146-9150 (1995)) or any other means of gene transfer known in the art. Vectors expressing gene products that interact with the target protein are isolated by identifying β-gal-positive clones. Similar systems employing β-lactamase are available (e.g., GeneBLAzer™ fusion vectors from Invitrogen, Carlsbad, Calif.). An advantage of these system is that the screen can be carried out in any cell type, regardless of the cell's milieu of endogenous (and potentially competing) proteins. A further possibility for this type of system is that the target protein can be localized to a specific cellular compartment, with the aim of identifying proteins involved in interactions restricted to that particular location. Fluorescence-activated cell sorting techniques can be used. For example, β-gal-positive cells which contain cDNAs expressing gene products that interact with the target protein will generate a signal that will allow such cells to be purified by cell-sorting techniques. Such cDNAs can be delivered, for example, using retroviral vectors that allow introduction of high complexity cDNA libraries with high infection efficiency.

Cell systems, such as described by Blau, can also be used to evaluate a wide variety of extracellular agents, such as for example, extracellular signaling molecules, growth factors or differentiation factors, peptides, drugs or synthetic analogs, or the like, whose presence or effects might alter the potential for interaction between two or more given proteins in a particular cell type. Thus, a cell including a reporter system can be used to screen test agents that can modulate the interaction of the putative binding proteins. This procedure can include contacting a cell with a test agent; contacting the cell with a liposome containing a substrate, as described herein, thereby introducing the substrate into the cell, wherein said substrate is capable of producing a detectable light signal when acted on by an enzyme. The cell includes a reporter system as described above. The screening procedure includes detecting the level of the light signal and comparing the level to a control cell not exposed to the test agent as an indication of the ability of the test agent to act as a modulator of interactions between the putative binding proteins. Non-limiting examples of test agents include antibiotics, anti-inflammatory drugs, neurotransmitters, growth hormones, and analgesics. Cells that include the reporter system including β-galatosidase are commercially available as the InteraX™ System available from Applied Biosystems, an Applera Corporation business (Foster City, Calif.). The InteraX™ system is a high-throughput screening (HTS) technology used to monitor specific functional protein/protein interactions in cellular compartments where they naturally occur. In some embodiments, the system can be applied to mammalian cell lines with intact regulatory machinery, which not only allows a functional readout for orphan and known G-Protein coupled receptors (GPCRs), but extend to a more general use of the technology as a tool to map signaling pathways. Cell-based assays that employ β-lactamase are available (e.g., PanVera® Cell-Based Assays, Invitrogen).

In some embodiments, the present enzyme detection methods and compositions can be used in conjunction with cells that include an expression construct comprising a transcriptional initiation regulatory region associated with a structural gene encoding a reporter enzyme such as β-galactosidase, β-lactamase, or luciferase (e.g., Coleoptera luciferase) which transforms a substrate to a detectable product as described herein (e.g., see U.S. Pat. Nos. 6,143,502; 5,700,673; 5,674,713; 5,583,024; and 5,070,012). In some embodiments, the expression construct comprises the transcriptional initiation regulatory region, the gene encoding an enzyme of interest, such as β-galactosidase, β-lactamase, or luciferase and a transcriptional termination region. The construct can be used to differentiate cells by virtue of a particular phenotype or genotype. The construct can be used to determine the efficiency of transfection or transformation or, alternatively, the efficiency of a particular promoter or enhancer or other transcriptional regulatory element, either by itself or in combination with other regulatory elements. The regulatory elements can be endogenous to the cell or can be as a result of the presence of a virus, for example. In some situations it can be desirable to determine the presence of a virus or virus particle in a physiological fluid or cell, either qualitatively or quantitatively. In some embodiments, the construct can be employed in determining the efficiency of infection where infection includes recombinant or defective retrovirus or similar virus infection; Epstein-Barr virus derived virus constructs leading to episomal maintenance; and transformation, where transformation includes transfection, transduction, or transformation employing various agents, such as electroporation, permeabilizing the membrane, for example with calcium phosphate, lysozyme, etc., or the like. The particular target host or DNA introduction procedure can be evaluated by comparing the fluorescence of the various cells which receive the DNA under different conditions.

In some embodiments, the methods herein can be used to detect an abnormality in the activity of an enzyme in a cell by comparing the activity observed using a test cell and a reference cell. The method can also be used to perform an assay for detecting a disease by determining the activity observed using a test cell and comparing the activity to a disease reference cell and a non-disease reference cell. Non-limiting examples of such disease or abnormal condition include cancer, infection, sepsis, immune disorders and anemia. In some embodiments, the enzyme assay can be used to study mutant forms of an enzyme. For example, the gene for an enzyme can be modified by conventional methods such as gene shuffling, site-directed mutagenesis and/or homologous recombination. The various mutant forms can be used to generate a library of cells, and the cells analyzed for the level of enzyme activity.

In the methods described herein, a fluorescence signal can be detected using conventional methods and instruments. In some embodiments, a multiwavelength fluorescence detector can be utilized. The detector can be used to excite the fluorescence labels at one wavelength and detect emissions as multiple wavelengths, or excite at multiple wavelengths and detect at one emission wavelength. Alternatively, the sample can be excited using “zero-order” excitation in which the full spectrum of light (e.g., from xenon lamp) illuminates the sample. Each label can absorb at its characteristic wavelength of light and then emit maximum fluorescence. The multiple emission signals can be detected independently. A suitable detector can be programmed to detect more than one excitation emission wavelength substantially simultaneously, such as that commercially available under the trade designation HP1100 (G1321A) (Hewlett Packard, Wilmington, Del.). Thus, the fluorescent products can be detected at programmed emission wavelengths at various intervals during a reaction.

In methods herein, cells are allowed to incubate for sufficient time so as to have a sufficient turnover of substrate to produce a light detectable signal. The signal can be observed in a variety of ways. For example, aliquots can be taken and used for fluorescence activated cell sorting (FACS), flow cytofluorometry or static cytofluorometry in a microscope or similar static device. In this manner, a distribution will be obtained for the various levels of fluorescence in the various cells, where the population acts in a heterogeneous manner. The total number of fluorescent cells can be determined where only a fraction of the total cells are infected to provide a particle count. Alternatively, or in combination, total fluorescence can be integrated at different times, so that an overall value can be obtained and the rate of change of the total fluorescence in the cells determined. The background value can be subtracted by employing controls, so that the increase in number of fluorescent cells and fluorescence per cell over time of the cell population can be determined and related to the factor of interest. Alternatively, the cells can be spread on a slide and a fluorescence microscope with an associated fluorometer employed to determine the level of fluorescence of individual cells or groups of cells (e.g., by epifluorescence microscopy). The particular manner in which fluorescence is determined for the cells in the assay is not critical and will vary depending upon available equipment, the qualitative or quantitative nature of the assay, and the like. General descriptions of cell sorting apparatus are provided in U.S. Pat. Nos. 4,172,227; 4,437,935; 4,661,913; 4,667,830; 5,093,234; 5,094,940; 5,144,224; and 6,566,508.

An example of a detection system useful in the present enzyme assay methods is the 8200 Cellular Detection System (Applied Biosystems, an Applera Corporation business). This system is a macro-confocal system based on fluorometric microvolume assay technology (FMAT) that utilizes laser scanning to excite fluorophore contained within cells. The system can differentiate between background fluorescence and that associated with cells and includes multiplexing and automated high-throughput capabilities.

Chemiluminescence can be detected using any of a variety of detectors. Non-limiting example of suitable detectors include luminometers (e.g, Veritas™ Microplate luminometer, Promega; TD-20/20 luminometer, Turner Design, Sunnyvale, Calif.; and BD Moonlight 3010 Luminometer, Becton-Dickinson Bioscience), a charge-couple device (CCD) camera, X-ray film, or a scintillation counter.

In some embodiments, light signals can be detected by visual inspection, colorimetry, light microscopy, digital image analyzing, standard microplate reader techniques, video cameras, photographic film. Data can be discriminated and/or analyzed by using pattern recognition software.

In another aspect, the present disclosure concerns kits for detecting an enzyme activity in a cell. The kit can comprise one or more of the following: liposomes; liposomes containing at least one substrate as described herein, wherein the substrate is capable of producing a light-detectable signal when acted on by an enzyme in a cell. Examples of such a signal include a fluorescent signal or a chemiluminescent signal. The liposomes can be neutral, cationic or anionic. In some embodiments, the liposomes of the kit can be included as a lyophilized preparation. In some embodiments, the liposomes are characterized in that cell viability is decreased by less than 20%, less than 10%, less than 5% or less than 1% when the liposomes are contacted with cells under conditions of an enzyme assay as described herein. The substrate can be essentially membrane impermeable. In some embodiments, the enzyme is β-galactosidase and the substrate is a β-galactosidyl substituted fluorogenic compound or substituted derivative thereof. In some embodiments, the substrate comprises 9H-(1,3-dichloro-9,9-dimethylacridin-2-one-7-yl) β-D-galactopyranoside. In some embodiments, the enzyme is caspase 3 and the substrate is a substrate of caspase 3. In some embodiments, the enzyme is β-lactamase. In some embodiments, the enzyme is luciferase. The kit can include a modulator (e.g., an inhibitor or an activator) of the enzyme. In one example, an inhibitor of β-galactosidase or of capase 3 is included. The cell can comprise a reporter system capable of detecting interactions between putative binding proteins. The kit can further include instructions for carrying an enzyme assay method as described herein. The kit can additionally include a cell or cell preparation, a reagent for determining cell viability, and media for suspending cells as described herein. In some embodiments, the media comprises serum. In some embodiment, the kit further comprises serum. In some embodiments, the serum is fetal bovine serum.

Unless defined otherwise, all technical and scientific terms used herein have the same meaning as commonly understood by one of ordinary skill in the art to which this disclosure belongs. Unless mentioned otherwise the techniques employed or contemplated herein are standard methodologies well known to one of ordinary skill in the art. The materials, methods and examples are illustrative only and not limiting.

All numerical ranges in this specification are intended to be inclusive of their upper and lower limits.

Other features of the disclosure will be come apparent in the course of the following descriptions of some embodiments which are given for illustration of the disclosure and are not intended to be limiting thereof.

### 7. EXAMPLES

Aspects of the present teachings may be further understood in light of the following examples, which should not be construed as limiting the scope of the present teachings in anyway.

### 7.1 Example 1

### Preparation of 100 nm Cationic Liposomes

Large unilamellar vesicles (LUV) of diameter 100 nm were prepared by the extrusion method essentially as described by Chatterjee, et al. (in Methods in Molecular Biology: Liposome Methods and Protocols (S. Basu and M. Basu eds.), Humana Press, 2002, vol. 199, chapter 1). Sterile techniques were used throughout this procedure to prevent bacterial contamination of the liposomes. 1,2-Dioleoyl-sn-Glycero-3-Phosphocholine (DOPC) (12.5 mg, Avanti Polar Lipids, catalog no. 850375), and 1,2-Dioleoyl-sn-Glycero-3-Ethylphosphocholine (EDOPC) (12.5 mg, Avanti Polar Lipids, catalog no. 890704) were dissolved in chloroform (5 ml) in a 25 ml recovery flask. The solvent was thoroughly evaporated under high vacuum to leave a thin film. Sterile filtered PBS buffer (2 ml, pH 7.2) was added and the suspension was subjected to five cycles of freezing (−78° C., dry ice acetone bath) under argon and thawing (40° C.) to hydrate the lipids. The resulting large multilamellar vesicles (LMV) were extruded ten times through two stacked 100 nm polycarbonate membranes (Nuclepore track-etch membrane, Whatman, catalog no. 110605) using a Lipex™ Extruder (Northern Lipids, Inc., British Columbia, Canada, catalog no. T.001). The LUV were purified by Sephadex™ G-25 M gel filtration (PD-10 column, Amersham Biosciences, catalog no. 17-0851-01) eluting with PBS. The liposome size and dispersity was determined by dynamic light scattering using a Nicomp 370 particle size analyzer (Lee Miller, Fine Particle Technology, Menlo Park, Calif.).

### 7.2 Example 2

### Preparation of 100 nm Cationic Liposomes Containing DDAO-gal or DDAO

DOPC and EDOPC were dissolved in chloroform and the solvent was evaporated to leave a thin film, as described in Example 1. 9H-(1,3-dichloro-9,9-dimethylacridin-2-one-7-yl) β-D-galactopyranoside (DDAO-gal) (5 mg, Molecular Probes, catalog no. D-6488) or 7-hydroxy-9H-(1,3-dichloro-9,9-dimethylacridin-2-one) (DDAO) (5 mg, Molecular Probes, catalog no. H-6482) was dissolved in DMSO (20 μl) and added to sterile filtered PBS buffer (2 ml, pH 7.2). The DDAO-gal or DDAO in PBS was added to the lipids and the suspension was subjected to five cycles of freezing (−78° C., dry ice acetone bath) under argon and thawing (40° C.) to hydrate the lipids. The resulting LMV were extruded and purified as described in Example 1. The concentration of DDAO-gal or DDAO within the liposomes was estimated to be 0.25 mg/ml.

### 7.3 Example 3

### Cell Staining with Liposomes Containing DDAO-gal

Psi 2 BAGα cells (ATCC, catalog no. CRL-9560) in Dulbecco's modified Eagle's medium (ATCC, catalog no. 30-2002) containing 5% Fetal Bovine Serum (FBS, HyClone, catalog no. SH30071.03) and 1% penicillin-streptomycin solution (pen/strep, ATCC, catalog no. 30-2300) were seeded (200 μl/well, 10,000 cell/well) in black 96 well microtiter plates (ABI, catalog no. 4308776). HeLa cells (ATCC, catalog no. CCL-2) in Eagle's minimum essential medium (ATCC, catalog no. 30-2003) containing 5% FBS and 1% pen/strep were seeded (200 μl/well, 10,000 cell/well) in black 96 well microtiter plates (ABI, catalog no. 4308776). CHO cells (ATCC, catalog no. CCL-61) in F-12K medium (ATCC, catalog no. 30-2004) containing 5% FBS and 1% pen/strep were seeded (200 μl/well, 10,000 cell/well) in black 96 well microtiter plates (ABI, catalog no. 4308776). After overnight incubation at 37° C. under 5% CO₂ the supernatants were removed carefully so as not to detach the adherent cells from the wells. Solutions of liposome encapsulated DDAO-gal, liposome encapsulated DDAO and nonencapsulated DDAO-gal were prepared in the appropriate cell medium and then added to the cells (200 μl/well). The nonencapsulated DDAO-gal solution was used as a negative control (no liposome, passive uptake) and the liposome encapsulated DDAO solution was used as a positive control (DDAO is the product of DDAO-gal hydrolysis). After incubation for 3 hr at 37° C. under 5% CO₂ the staining solutions were removed carefully and the cells were washed twice with Dulbecco's PBS (200 μl, ATCC, catalog no. 30-2200). PBS (200 μl) was added to the wells and the plate was read using an 8100 FMAT instrument (Applied Biosystems, an Applera Corporation business).

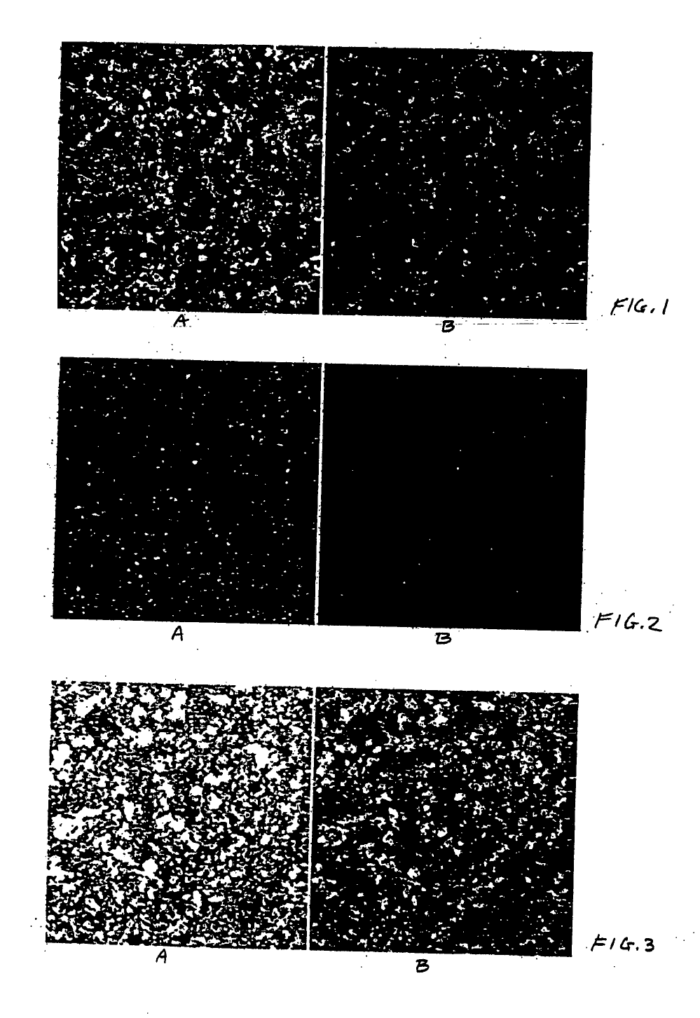

As shown in FIGS. 1A and 1B, when psi 2 BAGα cells were exposed to liposomes encapsulating the substrate DDAO-gal, fluorescent signal was detected. DDAO-gal without liposomes were used as a negative control (passive load) and liposomes containing DDAO were used as a positive control. FIG. 2 shows the fluorescent signal produced in cells exposed to DDAO-gal without liposomes. FIG. 3 shows the fluorescent signal produced in cells exposed to liposomes encapsulating DDAO. In each of FIGS. 1-9, the cells were excited using a Helium-Neon laser at 633 nm. Also, for each of FIGS. 1-9, panel A represents the emission data from 650-685 nm, and panel B represents the emission data from 685-710 nm.

Although DDAO-gal is intrinsically fluorescent (excitation/emission ˜460/610 nm), β-galactosidase-catalyzed hydrolysis of DDAO gal liberates the DDAO fluorophore, which absorbs and emits light at much longer wavelengths (excitation/emission ˜645/660 nm). Not only can DDAO be excited without interference from the substrate, but its fluorescence emission is detected at wavelengths that are well beyond the autofluorescence exhibited by most cellular samples.

Psi 2 BAGα cells constitutively express E. coli β-galactosidase. Contacting these cells with liposomes encapsulating DDAO-gal (FIG. 1B) led to the generation of a detectable fluorescent signal. When cells were exposed to DDAO-gal not contained within liposomes, a much lower signal was detected (FIG. 2B). The signal is much lower than that seen in FIG. 1B, demonstrating the ability of liposomes to introduce the substrate into the cells for cleavage by β-galactosidase enzyme. FIG. 3B shows the signal observed when encapsulated DDAO was contacted with cells. This emission was expected because the free dye emits at a wavelength of 660 nm.

The results shown in FIG. 1 demonstrated that contacting psi 2 BAGα cells with liposomes encapsulating the substrate DDAO-gal (FIG. 1B) led to the expected release of DDAO and that DDAO was retained within the cell.

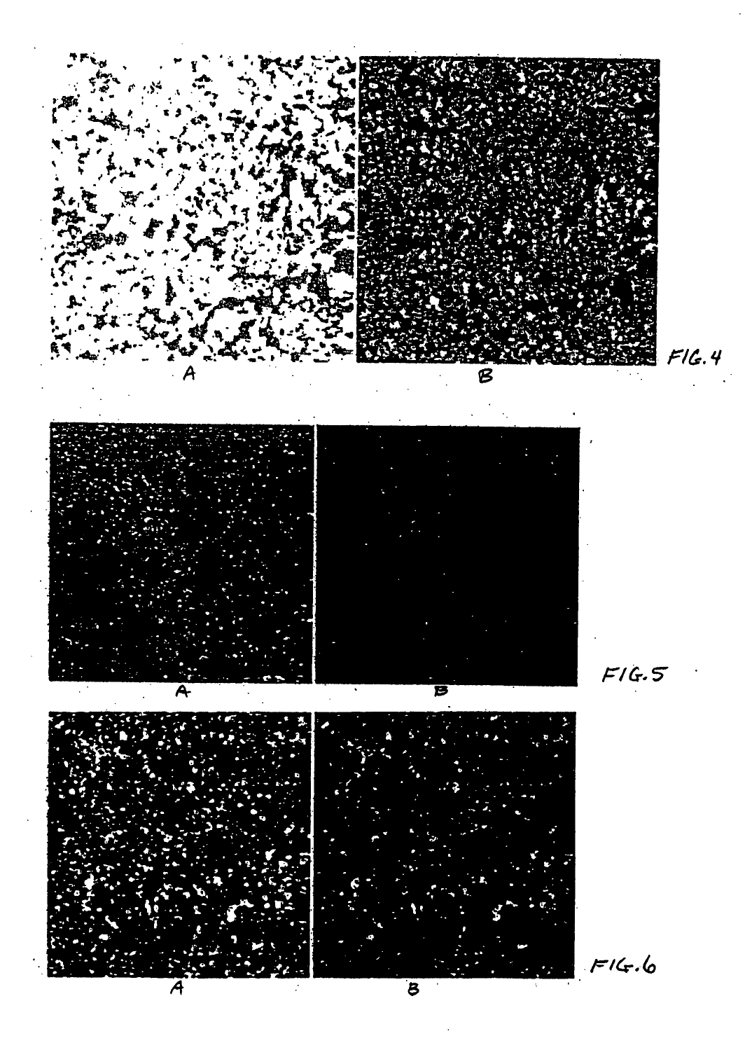

The experiments shown in FIGS. 4-6, were conducted under similar conditions to those of FIGS. 1-3, respectively, with the exception that HeLa cells were used. HeLa cells constitutively express a human form of β-galactosidase. The high fluorescent signal generated upon exposure of HeLa cells to liposomes encapsulating DDAO-gal (FIG. 4B) demonstrated that this form of β-galactosidase was active toward the substrate.

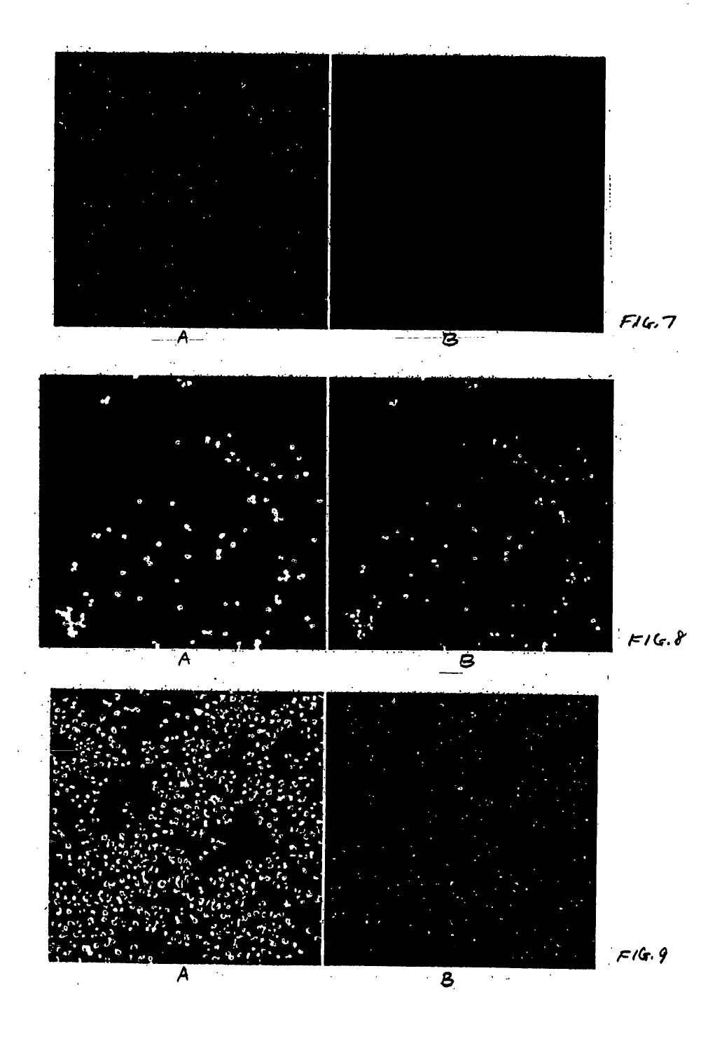

The experiments shown in FIGS. 7-9, were conducted under similar conditions to those of FIGS. 1-3, respectively, with the exception that Chinese Hamster Ovary (CHO) cells were used. CHO cells do not express endogenous β-galactosidase. The minimal fluorescent signal generated upon exposure of CHO cells to liposomes encapsulating DDAO-gal (FIG. 7B) demonstrated that these cells lacked active β-galactosidase. As a positive control, liposomes containing DDAO were contacted with CHO cells (FIG. 9), and signal was detected, as expected due to the fluorescence of the free dye. The signal observed when cells were exposed to DDAO-gal in the absence of liposomes was due to the emission by DDAO-gal that was passively delivered into the cells.

In conclusion, these results show that liposomes containing a substrate capable of generating a fluorescent signal when acted on by β-galactosidase can be used to detect activity of this enzyme in cells and can be used to determine the presence or absence of this activity in various cell types.

While the foregoing has presented some embodiments of the present teachings, it is to be understood that these embodiments have been presented by way of example only. It is expected that others will perceive and practice variations which, though differing from the foregoing do not depart form the spirit and scope of the present teachings as described and claimed herein.

Except those patent application cited in the “Cross-Reference To Related Applications” section above, all patent applications, patents, and literature references cited in this specification, are hereby incorporated by reference in their entirety. In the event that one or more of the incorporated literature and similar materials differ from or contradicts this application, including but not limited to defined terms, term usage, described techniques, or the like, this application controls. In this application, the use of the singular includes the plural unless specifically stated otherwise. “Comprise” and “comprises” are not intended to be limiting. The section headings used herein are for organization purposes only and are not to be construed as limiting the subject matter described in any way.

1. A method for detecting an activity of an enzyme in a cell, the method comprising:
contacting a cell with a liposome containing a substrate capable of producing a detectable light signal when acted upon by the enzyme, wherein said liposome facilitates intracellular delivery of said substrate; and
detecting a light signal in the cell that indicates the presence or amount of the enzyme activity in the cell.

2. A method of detecting interaction between putative binding proteins in a cell, the method comprising:
contacting a cell with a liposome containing a substrate capable of producing a detectable light signal when acted upon by an enzyme, wherein said liposome facilitates intracellular delivery of said substrate;
wherein said cell comprises a reporter system capable of detecting binding between putative binding proteins wherein said binding mediates the association of first and second reporter subunits to generate an enzymatically active complex capable of acting on said substrate to produce said signal; and
detecting said signal as an indication of said interaction.

3. A method of detecting interaction between putative binding proteins in a cell, the method comprising:
contacting a cell with a liposome containing a substrate of a reporter system wherein said liposome facilitates intracellular delivery of said substrate, wherein said substrate is capable of producing a detectable light signal when acted on by an enzyme,
wherein said cell comprises a reporter system comprising:
a) a first reporter subunit coupled to a first putative binding protein;
b) a second reporter subunit coupled to a second putative binding protein;
wherein said first reporter subunit and said second reporter subunit are capable of associating to generate an enzymatically active complex capable of acting on said substrate to produce said signal, wherein said associating is mediated by the binding of said first putative binding protein and said second putative binding protein; and
detecting said light signal as an indication of said interaction.

4. A screening method for determining the ability of a test agent to act as a modulator of interactions between binding proteins within a cell, said process comprising:
contacting a cell with a test agent;
contacting said cell with a liposome containing a substrate wherein said liposome facilitates intracellular delivery of said substrate, wherein said substrate is capable of producing a detectable light signal when acted on by an enzyme,
wherein said cell comprises a reporter system comprising:
a) a first reporter subunit coupled to a first putative binding protein;
b) a second reporter subunit coupled to a second putative binding protein;
wherein said first reporter subunit and said second reporter subunit are capable of associating to generate an enzymatically active complex capable of acting on said substrate to produce said signal, wherein said associating is mediated by the binding of said first putative binding protein and said second putative binding protein; and
detecting the level of said light signal and comparing said level to a control cell not exposed to said test agent as an indication of the ability of said test agent to act as a modulator of interactions.

5. A method for studying a regulatory element of a transcriptional initiation regulatory system in a viable mammalian cell, said method comprising:
contacting said cell with a liposome containing a substrate capable of producing a detectable light signal when acted upon by an enzyme, wherein said liposome facilitates intracellular delivery of said substrate,
wherein said cell comprises an expression construct comprising a transcriptional initiation regulatory region comprising a regulatory sequence of interest and a structural gene encoding said enzyme; and
detecting said light signal in said cell as a measure of the activity of said regulatory element.

6. A method as in any one of claims 1-5, in which said detectable signal is a fluorescent signal or a chemiluminescent signal.

7. A method as in any one of claims 1-6, further comprising monitoring the detectable light signal as a function of time.

8. A method as in any one of claims 1-5, in which said substrate is substantially membrane impermeable.

9. A method as in any one of claims 1-5, in which said liposome is neutral or anionic.

10. A method as in any one of claims 1-5, in which said liposome is cationic.

11. A method as in any one of claims 1-5, in which said liposome comprises 1,2-diacyl-sn-glycero-3-alkylphosphocholine.

12. The method of claim 11 in which the 1,2-diacyl-sn-glycero-3-alkylphosphocholine has the formula: 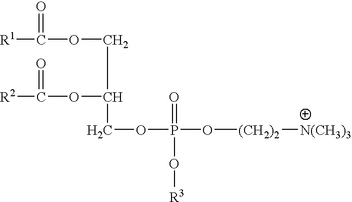
wherein:
R¹ is a saturated or unsaturated alkyl having from 6 to 30 carbon atoms;
R² is a saturated or unsaturated alkyl having from 6 to 30 carbon atoms;
R³ is a saturated or unsaturated alkyl having from 1 to 20 carbon atoms.

13. The method of claim 12 wherein said liposome comprises 1,2-dioleoyl-sn-glycero-3-ethylphosphocholine.

14. The method of claim 11 wherein said liposome comprises 1,2-dioleoyl-sn-glycero-3-ethylphosphocholine and 1,2-dioleoyl-sn-glycero-3-phosphocholine.

15. The method of claim 14 wherein said 1,2-dioleoyl-sn-glycero-3-ethylphosphocholine and said 1,2-dioleoyl-sn-glycero-3-phosphocholine are present in a molar ratio of about 1:1.

16. A method as in any one of the preceding claims, in which said liposome comprises cholesterol.

17. The method of claim 16 wherein said liposome comprises a unilamellar liposome with a diameter in the range of about 50 nm to about 250 nm.

18. A method as in any one of claims 1-7, in which the substrate comprises a β-galactosidyl substituted fluorogenic compound or substituted derivative thereof.

19. A method as in any one of claims 1-7, in which the substrate comprises a α-galactosidyl substituted fluorescein or substituted derivative thereof.

20. A method as in any one of claims 1-7, in which said substrate comprises at least one of 9H-(1,3-dichloro-9,9-dimethylacridin-2-one-7-yl) β-D-galactopyranoside, fluorescein di-β-D-galactoside, 2-nitrophenyl β-D-galactopyranoside, resorufin β-D-galactopyranoside, 6,8-difluoro-4-methylumbelliferyl β-D-galactopyranoside, β-methylumbelliferyl β-D-galactopyranoside, 3-carboxyumbelliferyl β-D-galactopyranoside, 5-chloromethylfluorescein di-β-D-galactopyranoside, 5-bromo-4-chloro-3-indoyl-β-galactopyranoside and 5-(pentafluorobenzoylamino)fluorescein di-β-D-galactopyranoside.

21. A method as in any one of claims 1-7, in which the substrate comprises 9H-(1,3-dichloro-9,9-dimethylacridin-2-one-7-yl) β-D-galactopyranoside.

22. A method as in any one of claims 1-7, in which said substrate comprises a FRET dye pair.

23. A method as in any one of claims 1-7, in which said substrate comprises at least one of 5-thia-1-azabicyclo[4.2.0]oct-2-ene-2-carboxylic acid, 8-oxo-3-[3-[(2-oxo-2H-1-benzopyran-7-yl)oxy]-1-propenyl]-7-[(phenylacetyl)amino]-(6R,7R)-(9CI), 5-thia-1-azabicyclo[4.2.0]oct-2-ene-2-carboxylic acid, 8-oxo-3-[3-[(2-oxo-2H-1-benzopyran-7-yl)oxy]-1-propenyl]-7-[(phenylacetyl)amino]-5-oxide (6R,7R)-(9CI),
5-thia-1-azabicyclo[4.2.0]oct-2-ene-2-carboxylic acid, 8-oxo-3-[(1Z)-3-[(3-oxo-3H-phenoxazin-7-yl)oxy]-1-propenyl]-7-[(2-thienylacetyl)amino]-5-oxide (6R,7R)-(9CI),
5-thia-1-azabicyclo[4.2.0]oct-2-ene-2-carboxylic acid, 7-[[[[(6-chloro-7-hydroxy-2-oxo-2H-1-benzopyran-3-yl)carbonyl]amino]acetyl]amino]-3-[[(3′,6′-dihydroxy-3-oxospiro[isobenzofuran-1 (3H), 9′-[9H]xanthen]-5-yl)thio]methyl]-8-oxo (6R,7R)-(9CI),
5-thia-1-azabicyclo[4.2.0]oct-2-ene-2-carboxylic acid, 8-oxo-3-[3-[(3-oxo-3H-phenoxazin-7-yl)oxy]-1-propenyl]-7-[(2-thienylacetyl)amino]-(acetyloxy)methyl ester, 5-oxide (6R,7R)-(9CI), and
5-thia-1-azabicyclo[4.2.0]oct-2-ene-2-carboxylic acid, 7-[[[[[7-[(acetyloxy)methoxy]-6-chloro-2-oxo-2H-1-benzopyran-3-yl]carbonyl]amino]acetyl]amino]-3-[[[3′,6′-bis(acetyloxy)-3-oxospiro[isobenzofuran-1 (3H), 9′-[9H]xanthen]-5-yl]thio]methyl]-8-oxo-, (acetyloxy)methyl ester (6R,7R)-(9CI).

24. A method as in any one of claims 1-7, in which said substrate comprises at least one of varglin luciferin, coelenterazine, firefly luciferin, cyprinda luciferin, bacterial luciferin, dinoflagellate luciferin, luciferin 6′ chloroethyl ether, luciferin 6′ methyl ether, 6′ deoxyluciferin, and luciferin 6′ benzyl ether.

25. A method as in any one of claims 1-7, in which said substrate comprises at least one of a peptide identified by SEQ ID:1, SEQ ID:2, SEQ ID:3 and SEQ ID:4.

26. A method as in any one of the preceding claims, in which said substrate is present in said liposome at a concentration in the range of about 1 nM to 100 mM.

27. A method as in any one of the preceding claims, in which said substrate is provided in a form that is at least 90% liposomal.

28. A method as in any one of claims 1-7, in which said enzyme comprises at least one enzyme selected from β-glucuronidase, carboxylesterase, lipases, phospholipases, sulphatases, ureases, peptidases, sulfatases, thioesterases, proteases, alkaline phosphatases, acid phosphatases, esterases, decarboxylases, phospholipase D,P-xylosidase, β-fucosidase, thioglucosidase, α-galactosidase, α-glucosidase, β-glucosidase, α-glucuronidase, α-mannosidase, α-mannosidase, α-fructofuranosidase, β-glucosiduronase, trypsin, hydrolases, oxidoreductases, saccharidases, β-glucosidase, α-lactamases, β-hexosaminidase, cholesterol esterase, nucleases, arylsulfatase, phospholipase, caspase 3, and luciferase.

29. A method as in any one of claims 1-7, in which said enzyme comprises at least one enzyme selected from hydrolases, oxidoreductases, saccharidases, β-glucosidase, β-lactamases, β-glucuronidase, α-galactosidase, β-hexosaminidase, esterases, cholesterol esterase, arylsulfatase, phospholipase, caspase 3, and phosphatase.

30. A method as in any one of claims 1-7, in which said enzyme comprises β-galactosidase.

31. A method as in any one of claims 1-7, in which said enzyme comprises β-lactamase.

32. A method as in any one of claims 1-7, in which said enzyme comprises E. coli β-glucosidase or E. coli TME-1 β-lactamase.

33. A method as in any one of claims 1-7, in which said enzyme comprises luciferase.

34. A method as in any one of claims 1-7, in which said enzyme comprises firefly luciferase or Rinella luciferase.

35. A method according to claims 1, in which said enzyme comprises at least one enzyme selected from estrogen sulfotransferase (SULT 1E), farnesyl:protein transferase, sialyl transferase, histone deacetylase, caspase 8, protein kinase A, protein kinase C, protein kinase Cα, protein kinase Cβ, protein kinase Cγ, chloramphenicol acetyltransferase, secreted form of human placental alkaline phosphatase, glutathione-S-transferase, uricase, β-glucuronidase and cytochrome P450.

36. A method according to claims 1, in which said enzyme comprises at least one enzyme selected from protein kinases, estrogen sulfotransferases, carbohydrate sulfotransferases, tyrosylprotein sulfotransferases, farnesyl transferases, COX-1, COX-2, dihydrofolate reductase, cytochrome P450, aromatase, alcohol dehydrogenase, acetylcholinesterase, sialyl transferase, adenylyl cyclase, inositol phosphoceramide (IPC) synthase, glycosyl transferases, lanosterol 14α-demethylase, type 2 fatty acid synthase, thymidylate synthase, geranylgeranyl transferase, methionine synthase, serine hydroxymethyltransferase, HMG-CoA reductase, histone acetyltransferase, histone deacetylase, cyclic nucleotide phosphodiesterases, phosphoinositide 3 kinase, 17β-hydroxysteroid dehydrogenase, topoisomerase, telomerase, squalene synthase, palmatoyl transferase, myristoyl transferase, luciferase and caspase 8.

37. A method according to claim 1, in which said liposome contains at least two substrates, wherein said substrates are capable of producing a detectable light signal, and wherein the signal from each substrate is distinguishable from the other.

38. A method according to claim 1, in which said cell is contacted with a modulator of said enzyme.

39. A method according to claim 1, in which said liposome includes a modulator of said enzyme.

40. A method as in any one of the preceding claims, in which said liposome includes a fluorescent tracer compound for confirming introduction of said substrate into said cell.

41. A method as in any one of the preceding claims, in which said cell is a mammalian or yeast cell.

42. A method as in any one of the preceding claims, in which said cell is a mammalian cell and is suspended in a medium comprising serum.

43. A method as in any one of the preceding claims, in which the viability of cells during said contacting decreases by less than about 20%.

44. A method as in any one of the preceding claims, in which the temperature during said contacting and said detecting is greater than a membrane freezing point of said cell.

45. A method as in any one of claims 1-6, in which said detecting is by means of a cell sorter or flow cytometer.

46. A method as in any one of claims 1-6, in which said detecting is by means of a fluorometer, fluorescence microscope, or laser scanning confocal microscope.

47. A method as in any one of claims 1-6, in which said detecting comprises analyzing said cell by epifluorescense microscopy.

48. A method as in any one of claims 1-7, in which said detecting is by means of a luminometer, CCD camera, or scintillation counter.

49. A method according to claim 4, in which said test agent is an antagonist of said interactions.

50. The method of claim 4 wherein said test agent is an agonist of said interactions.

51. The method of claim 4 wherein said test agent and said substrate are contained within said liposome during said contacting.

52. The method of claim 4 wherein said test agent is an antibiotic, an anti-inflammatory drug, a neurotransmitter, a growth hormone, or an analgesic.

53. A composition for use in detecting the activity of an enzyme in a cell, the composition comprising:
liposomes containing a substrate, wherein said substrate is capable of producing a detectable light signal when acted on by an enzyme in a cell, wherein said liposomes are characterized by causing less than about a 20% loss in viability of cells contacted with said liposomes.

54. The composition of claim 53 wherein said liposomes are characterized by causing less than about a 5% loss in viability of cells contacted with said liposomes

55. The composition claim 53 wherein said liposome contains at least two substrates, wherein said substrates are capable of producing a detectable light signal, and wherein the signal from each substrate is distinguishable from the other.

56. The composition of claim 53 wherein said substrate is substantially membrane impermeable.

57. The composition of claim 53 wherein said liposome is neutral or anionic.

58. The composition of claim 53 wherein said liposome is cationic.

59. The composition of claim 53 wherein said liposome comprises 1,2-diacyl-sn-glycero-3-alkylphosphocholine.

60. The composition of claim 59 in which the 1,2-diacyl-sn-glycero-3-alkylphosphocholine has the formula: 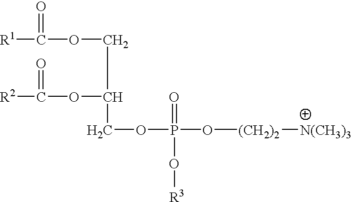
wherein:
R¹ is a saturated or unsaturated alkyl having from 6 to 30 carbon atoms;
R² is a saturated or unsaturated alkyl having from 6 to 30 carbon atoms;
R³ is a saturated or unsaturated alkyl having from 1 to 20 carbon atoms.

61. The composition of claim 60 wherein said liposome comprises 1,2-dioleoyl-sn-glycero-3-ethylphosphocholine.

62. The composition of claim 60 wherein said liposome comprises 1,2-dioleoyl-sn-glycero-3-ethylphosphocholine and 1,2-dioleoyl-sn-glycero-3-phosphocholine.

63. The composition of claim 62 wherein said 1,2-dioleoyl-sn-glycero-3-ethylphosphocholine and said 1,2-dioleoyl-sn-glycero-3-phosphocholine are present in a molar ratio of about 1:1.

64. The composition of claim 53 in which said liposome comprises cholesterol.

65. The composition of claim 53 wherein said liposome comprises a unilamellar liposome with a diameter in the range of about 50 nm to about 250 nm.

66. The composition of claim 53 in which the substrate comprises a β-galactosidyl substituted fluorogenic compound or substituted derivative thereof.

67. The composition of claim 53 in which the substrate comprises a β-galactosidyl substituted fluorescein or substituted derivative thereof.

68. The composition of claim 53 in which said substrate comprises at least one of 9H-(1,3-dichloro-9,9-dimethylacridin-2-one-7-yl) β-D-galactopyranoside, fluorescein di-β-D-galactoside, 2-nitrophenyl β-D-galactopyranoside, resorufin β-D-galactopyranoside, 6,8-difluoro-4-methylumbelliferyl β-D-galactopyranoside, β-methylumbelliferyl β-D-galactopyranoside, 3-carboxyumbelliferyl β-D-galactopyranoside, 5-chloromethylfluorescein di-β-D-galactopyranoside, 5-bromo-4-chloro-3-indoyl-β-galactopyranoside and 5-(pentafluorobenzoylamino)fluorescein di-β-D-galactopyranoside.

69. The composition of claim 53 in which the substrate comprises 9H-(1,3-dichloro-9,9-dimethylacridin-2-one-7-yl) β-D-galactopyranoside.

70. The composition of claim 53 in which said substrate comprises a FRET dye pair.

71. The composition of claim 53 in which said substrate comprises at least one of 5-thia-1-azabicyclo[4.2.0]oct-2-ene-2-carboxylic acid, 8-oxo-3-[3-[(2-oxo-2H-1-benzopyran-7-yl)oxy]-1-propenyl]-7-[(phenylacetyl)amino]-(6R,7R)-(9CI),
5-thia-1-azabicyclo[4.2.0]oct-2-ene-2-carboxylic acid, 8-oxo-3-[3-[(2-oxo-2H-1-benzopyran-7-yl)oxy]-1-propenyl]-7-[(phenylacetyl)amino]-5-oxide (6R,7R)-(9CI),
5-thia-1-azabicyclo[4.2.0]oct-2-ene-2-carboxylic acid, 8-oxo-3-[(1Z)-3-[(3-oxo-3H-phenoxazin-7-yl)oxy]-1-prop enyl]-7-[(2-thienylacetyl)amino]-5-oxide (6R,7R)-(9 CI),
5-thia-1-azabicyclo[4.2.0]oct-2-ene-2-carboxylic acid, 7-[[[[(6-chloro-7-hydroxy-2-oxo-2H-1-benzopyran-3-yl)carbonyl]amino]acetyl]amino]-3-[[(3′,6′-dihydroxy-3-oxospiro[isobenzofuran-1 (3H), 9′-[9H]xanthen]-5-yl)thio]methyl]-8-oxo (6R,7R)-(9CI),
5-thia-1-azabicyclo[4.2.0]oct-2-ene-2-carboxylic acid, 8-oxo-3-[3-[(3-oxo-3H-phenoxazin-7-yl)oxy]-1-propenyl]-7-[(2-thienylacetyl)amino]-(acetyloxy)methyl ester, 5-oxide (6R,7R)-(9CI), and
5-thia-1-azabicyclo[4.2.0]oct-2-ene-2-carboxylic acid, 7-[[[[[7-[(acetyloxy)methoxy]-6-chloro-2-oxo-2H-1-benzopyran-3-yl]carbonyl]amino]acetyl]amino]-3-[[[3′,6′-bis(acetyloxy)-3-oxospiro[isobenzofuran-1(3H), 9′-[9H]xanthen]-5-yl]thio]methyl]-8-oxo-, (acetyloxy)methyl ester (6R,7R)-(9CI).

72. The composition of claim 53 in which said substrate comprises at least one of varglin luciferin, coelenterazine, firefly luciferin, cyprinda luciferin, bacterial luciferin, and dinoflagellate luciferin.

73. The composition of claim 53 in which said substrate comprises at least one of luciferin 6′ chloroethyl ether, luciferin 6′ methyl ether, 6′ deoxyluciferin, and luciferin 6′ benzyl ether.

74. The composition of claim 53 in which said substrate is present in said liposome at a concentration of about 1 nM to 10 mM.

75. The composition of claim 53 in which said substrate is provided in a form that is at least 90% liposomal.

76. The composition of claim 53 in which said enzyme comprises at least one enzyme selected from β-glucuronidase, carboxylesterase, lipases, phospholipases, sulphatases, ureases, peptidases, sulfatases, thioesterases, proteases, alkaline phosphatases, acid phosphatases, esterases, decarboxylases, phospholipase D, β-xylosidase, β-fucosidase, thioglucosidase, α-galactosidase, α-glucosidase, β-glucosidase, β-glucuronidase, α-mannosidase, β-mannosidase, β-fructofuranosidase, β-glucosiduronase, trypsin, hydrolases, oxidoreductases, saccharidases, α-glucosidase, β-lactamases, β-hexosaminidase, cholesterol esterase, nucleases, arylsulfatase, phospholipase, caspase 3, and luciferase.

77. The composition of claim 53 in which said enzyme comprises at least one enzyme selected from hydrolases, oxidoreductases, saccharidases, β-glucosidase, α-lactamases, β-glucuronidase, α-galactosidase, β-hexosaminidase, esterases, cholesterol esterase, arylsulfatase, phospholipase, caspase 3, and phosphatase.

78. The composition of claim 53 in which said enzyme comprises β-galactosidase.

79. The composition of claim 53 in which said enzyme comprises α-lactamase.

80. The composition of claim 53 in which said enzyme is E. coli β-glucosidase or E. coli TME-1 β-lactamase.

81. The composition of claim 53 in which said enzyme comprises luciferase.

82. The composition of claim 53 in which said enzyme is firefly luciferase or Rinella luciferase.

83. The composition of claim 53 in which said enzyme comprises at least one enzyme selected from estrogen sulfotransferase (SULT 1E), farnesyl:protein transferase, sialyl transferase, histone deacetylase, caspase 8 and cytochrome P450.

84. The composition of claim 53 in which said enzyme comprises at least one enzyme selected from protein kinases, estrogen sulfotransferases, carbohydrate sulfotransferases, tyrosylprotein sulfotransferases, farnesyl transferases, COX-1, COX-2, dihydrofolate reductase, cytochrome P450, aromatase, alcohol dehydrogenase, acetylcholinesterase, sialyl transferase, adenylyl cyclase, inositol phosphoceramide (IPC) synthase, glycosyl transferases, lanosterol 14α-demethylase, type 2 fatty acid synthase, thymidylate synthase, geranylgeranyl transferase, methionine synthase, serine hydroxymethyltransferase, HMG-CoA reductase, histone acetyltransferase, histone deacetylase, cyclic nucleotide phosphodiesterases, phosphoinositide 3 kinase, 17β-hydroxysteroid dehydrogenase, topoisomerase, telomerase, squalene synthase, palmatoyl transferase, myristoyl transferase, luciferase and caspase 8.

85. The composition of claim 53 in which said liposome contains at least two substrates, wherein said substrates are capable of producing a detectable light signal, and wherein the signal from each substrate is distinguishable from the other.

86. The composition of claim 53 in which said cell is contacted with a modulator of said enzyme.

87. The composition of claim 53 in which said liposome includes a modulator of said enzyme.

88. The composition of claim 53 in which said liposome includes a fluorescent tracer compound for confirming introduction of said substrate into said cell.

89. The composition of claim 53 in which said cell is a mammalian, bacterial, or yeast cell.

90. The composition of claim 53 in which said cell is a mammalian cell and is suspended in medium comprising serum.

91. The composition of claim 53 wherein said detectable signal is a fluorescent signal or a chemiluminescent signal.

92. The composition of claim 53 wherein said cell is a mammalian, bacterial or yeast cell.

93. The composition of claim 53 wherein said cell comprises a reporter system capable of detecting interactions between putative binding proteins.

94. The composition of claim 53 further including a modulator of interactions between putative binding proteins.

95. The composition of claim 53 further including medium suitable for incubating said cell wherein said medium comprises serum and wherein said cell is a mammalian cell.

96. The composition of claim 95 wherein said medium comprises fetal bovine serum.

97. The composition of claim 95 wherein said serum is present at a concentration in the range of about 3% to about 7%

98. The composition of claim 97 wherein said serum is present at a concentration of about 5%.

99. A kit for use in detecting an enzyme activity in a cell, the kit comprising: liposomes containing a substrate, wherein said substrate is capable of producing a detectable light signal when acted on by an enzyme in a cell, wherein said liposomes are characterized in that at least 90% of cells exposed to said liposomes remain viable.

100. The kit of claim 99 wherein said liposome is neutral or anionic.

101. The kit of claim 99 wherein said liposome is cationic.

102. The kit of claim 99, in which said liposome comprises 1,2-diacyl-sn-glycero-3-alkylphosphocholine.

103. The kit of claim 102 in which the 1,2-diacyl-sn-glycero-3-alkylphosphocholine has the formula: 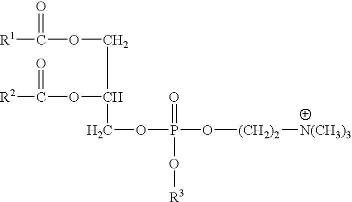
wherein:
R¹ is a saturated or unsaturated alkyl having from 6 to 30 carbon atoms;
R² is a saturated or unsaturated alkyl having from 6 to 30 carbon atoms;
R³ is a saturated or unsaturated alkyl having from 1 to 20 carbon atoms.

104. The kit of claim 103 wherein said liposome comprises 1,2-dioleoyl-sn-glycero-3-ethylphosphocholine.

105. The kit of claim 99 wherein said liposome comprises 1,2-dioleoyl-sn-glycero-3-ethylphosphocholine and 1,2-dioleoyl-sn-glycero-3-phosphocholine.

106. The kit of claim 105 wherein said 1,2-dioleoyl-sn-glycero-3-ethylphosphocholine and said 1,2-dioleoyl-sn-glycero-3-phosphocholine are present in a molar ratio of about 1:1.

107. A kit as in any one of claims 99-106, in which said liposome comprises cholesterol.

108. The kit of any one of claims 99-107, in which said liposome comprises a unilamellar liposome with a diameter in the range of about 50 nm to about 250 nm.

109. The kit of claim 99, in which the substrate comprises a β-galactosidyl substituted fluorogenic compound or substituted derivative thereof.

110. The kit of claim 99, in which the substrate comprises a β-galactosidyl substituted fluorescein or substituted derivative thereof.

111. The kit of claim 99, in which said substrate comprises at least one of 9H-(1,3-dichloro-9,9-dimethylacridin-2-one-7-yl) β-D-galactopyranoside, fluorescein di-β-D-galactoside, 2-nitrophenyl β-D-galactopyranoside, resorufin β-D-galactopyranoside, 6,8-difluoro-4-methylumbelliferyl β-D-galactopyranoside, β-methylumbelliferyl β-D-galactopyranoside, 3-carboxyumbelliferyl β-D-galactopyranoside, 5-chloromethylfluorescein di-β-D-galactopyranoside, 5-bromo-4-chloro-3-indoyl-β-galactopyranoside and 5-(pentafluorobenzoylamino)fluorescein di-β-D-galactopyranoside.

112. The kit of claim 99, in which the substrate comprises 9H-(1,3-dichloro-9,9-dimethylacridin-2-one-7-yl) β-D-galactopyranoside.

113. The kit of claim 99, in which said substrate comprises a FRET dye pair.

114. The kit of claim 99, in which said substrate comprises at least one of 5-thia-1-azabicyclo[4.2.0]oct-2-ene-2-carboxylic acid, 8-oxo-3-[3-[(2-oxo-2H-1-benzopyran-7-yl)oxy]-1-propenyl]-7-[(phenylacetyl)amino]-(6R,7R)-(9CI),
5-thia-1-azabicyclo[4.2.0]oct-2-ene-2-carboxylic acid, 8-oxo-3-[3-[(2-oxo-2H-1-benzopyran-7-yl)oxy]-1-propenyl]-7-[(phenylacetyl)amino]-5-oxide (6R,7R)-(9CI),
5-thia-1-azabicyclo[4.2.0]oct-2-ene-2-carboxylic acid, 8-oxo-3-[(1Z)-3-[(3-oxo-3H-phenoxazin-7-yl)oxy]-1-propenyl]-7-[(2-thienylacetyl)amino]-5-oxide (6R,7R)-(9CI),
5-thia-1-azabicyclo[4.2.0]oct-2-ene-2-carboxylic acid, 7-[[[[(6-chloro-7-hydroxy-2-oxo-2H-1-benzopyran-3-yl)carbonyl]amino]acetyl]amino]-3-[[(3′,6′-dihydroxy-3-oxospiro[isobenzofuran-1 (3H), 9′-[9H]xanthen]-5-yl)thio]methyl]-8-oxo (6R,7R)-(9CI),
5-thia-1-azabicyclo[4.2.0]oct-2-ene-2-carboxylic acid, 8-oxo-3-[3-[(3-oxo-3H-phenoxazin-7-yl)oxy]-1-propenyl]-7-[(2-thienylacetyl)amino]-(acetyloxy)methyl ester, 5-oxide (6R,7R)-(9CI), and
5-thia-1-azabicyclo[4.2.0]oct-2-ene-2-carboxylic acid, 7-[[[[[7-[(acetyloxy)methoxy]-6-chloro-2-oxo-2H-1-benzopyran-3-yl]carbonyl]amino]acetyl]amino]-3-[[[3′,6′-bis(acetyloxy)-3-oxospiro[isobenzofuran-1 (3H), 9′-[9H]xanthen]-5-yl]thio]methyl]-8-oxo-, (acetyloxy)methyl ester (6R,7R)-(9CI).

115. The kit of claim 99, in which said substrate comprises at least one of varglin luciferin, coelenterazine, firefly luciferin, cyprinda luciferin, bacterial luciferin, and dinoflagellate luciferin.

116. The kit of claim 99, in which said substrate comprises at least one of luciferin 6′ chloroethyl ether, luciferin 6′ methyl ether, 6′ deoxyluciferin, and luciferin 6′ benzyl ether.

117. A kit as in any one of claims 109-116, in which said substrate is present in said liposome at a concentration of about 1 nM to 10 mM.

118. The kit of claim 99 wherein said substrate comprises a substrate of caspase 3.

119. A kit as in any one of claims 109-118, in which said substrate is provided in a form that is at least 90% liposomal.

120. The kit of claim 99, in which the detectable signal is a fluorescent signal or a chemiluminescent signal.

121. The kit of claim 99 wherein said substrate is substantially membrane impermeable.

122. The kit of claim 99, in which said enzyme comprises an enzyme selected from β-glucuronidase, carboxylesterase, lipases, phospholipases, sulphatases, ureases, peptidases, sulfatases, thioesterases, proteases, alkaline phosphatases, acid phosphatases, esterases, decarboxylases, phospholipase D,P-xylosidase, β-fucosidase, thioglucosidase, α-galactosidase, α-glucosidase, β-glucosidase, β-glucuronidase, α-mannosidase, β-mannosidase, β-fructofuranosidase, β-glucosiduronase, trypsin, hydrolases, oxidoreductases, saccharidases, β-glucosidase, β-lactamases, α-hexosaminidase, cholesterol esterase, nucleases, arylsulfatase, phospholipase, caspase 3, and luciferase.

123. The kit of claim 99, in which said enzyme comprises an enzyme selected from hydrolases, oxidoreductases, saccharidases, β-glucosidase, β-lactamases, β-glucuronidase, α-galactosidase, β-hexosaminidase, esterases, cholesterol esterase, arylsulfatase, phospholipase, caspase 3, and phosphatase.

124. The kit of claim 99, in which said enzyme comprises β-galactosidase.

125. The kit of claim 99, in which said enzyme comprises β-lactamase.

126. The kit of claim 99, in which said enzyme comprises at least one enzyme selected from E. coli β-glucosidase and E. coli TME-1 β-lactamase.

127. The kit of claim 99, in which said enzyme comprises luciferase.

128. The kit of claim 99, in which said enzyme comprises at least one enzyme selected from firefly luciferase and Rinella luciferase.

129. The kit of claim 99, in which said enzyme comprises at least one enzyme selected from estrogen sulfotransferase (SULT 1E), farnesyl:protein transferase, sialyl transferase, histone deacetylase, caspase 8 and cytochrome P450.

130. The kit of claim 99, in which said enzyme comprises at least one enzyme selected from protein kinases, estrogen sulfotransferases, carbohydrate sulfotransferases, tyrosylprotein sulfotransferases, farnesyl transferases, COX-1, COX-2, dihydrofolate reductase, cytochrome P450, aromatase, alcohol dehydrogenase, acetylcholinesterase, sialyl transferase, adenylyl cyclase, inositol phosphoceramide (IPC) synthase, glycosyl transferases, lanosterol 14α-demethylase, type 2 fatty acid synthase, thymidylate synthase, geranylgeranyl transferase, methionine synthase, serine hydroxymethyltransferase, HMG-CoA reductase, histone acetyltransferase, histone deacetylase, cyclic nucleotide phosphodiesterases, phosphoinositide 3 kinase, 17β-hydroxysteroid dehydrogenase, topoisomerase, telomerase, squalene synthase, palmatoyl transferase, myristoyl transferase, luciferase and caspase 8.

131. The kit of claim 99, in which said liposome contains at least two substrates, wherein said substrates are capable of producing a detectable light signal, and wherein the signal from each substrate is distinguishable from the other.

132. The kit of claim 99, in which said cell is contacted with a modulator of said enzyme.

133. The kit of claim 99, in which said liposome includes a modulator of said enzyme.

134. The kit of claim 99, in which said liposome includes a fluorescent tracer compound for confirming introduction of said substrate into said cell.

135. The kit of claim 99, in which said cell is a mammalian or a yeast cell.

136. The kit of claim 135, in which said cell is a mammalian cell and is suspended in a medium comprising serum.

137. The kit of claim 99 wherein said detectable signal is a fluorescent signal or a chemiluminescent signal.

138. The kit of claim 99 wherein said cell comprises a reporter system capable of detecting interactions between putative binding proteins.

139. The kit of claim 138 further including a modulator of interactions between putative binding proteins.

140. The kit of claim 99 further including medium suitable for incubating said cell wherein said medium comprises serum and wherein said cell is a mammalian cell.

141. The kit of claim 140 wherein said medium comprises fetal bovine serum.

142. The kit of claim 138 wherein said serum is present at a concentration in the range of about 3% to about 7%

143. The kit of claim 140 wherein said serum is present at a concentration of about 5%.
